(19) United States
(12) Patent Application Publication
(10) Pub. No.: US 2026/0047968 A1
(43) Pub. Date: Feb. 19, 2026

(54) SYSTEMS AND METHODS FOR CONTROLLING OPERATIONS OF AN ARTICLE OF FURNITURE

(71) Applicant: Eight Sleep Inc., New York, NY (US)

(72) Inventors: Wesley BLUMMER, Oakland, CA (US); Jaewon Samuel KANG, Oakland, CA (US); Harry Chen XIAO, Redwood City, CA (US); Zachary WHITTLES, San Mateo, CA (US); Alexander Igorevich LEDNEV, Forest Park, IL (US); David HEINZ, San Francisco, CA (US); Daniel David SACHS, San Francisco, CA (US); Paras KANSRA, New York, NY (US); Angela Junyan CHU, San Francisco, CA (US); Matteo FRANCESCHETTI, Miami Shores, FL (US)

(21) Appl. No.: 19/365,921

(22) Filed: Oct. 22, 2025

Related U.S. Application Data
(63) Continuation of application No. PCT/US24/58951, filed on Dec. 6, 2024.
(60) Provisional application No. 63/607,162, filed on Dec. 7, 2023.
(60) Provisional application No. 63/548,598, filed on Feb. 1, 2024.
(60) Provisional application No. 63/644,264, filed on May 8, 2024.
(60) Provisional application No. 63/644,422, filed on May 8, 2024.

(51) Int. Cl. A61G 7/05; A47C 21/04; A61G 7/012

(52) U.S. Cl. CPC A61G 7/05; A47C 21/042; A47C 21/048; A61G 7/012; A61G 2203/16; A61G 2210/70; A61G 2210/90

(57) ABSTRACT

A system comprises a sensor zone comprising a cover layer comprising a first surface and a second surface that are opposite from one another. The first surface of the cover layer comprises a textured surface. A sensor unit is disposed on or adjacent to the second surface. The sensor unit is configured to detect a user's contacting the textured surface, and generate a digital data indicative of the detection. The system comprises a complex comprising one or more fluidic channels (e.g., tubes) disposed within one or more grooves of a support member (e.g., foam). The system comprises one or more sensors. The one or more sensor(s) is disposed over, in, or under the support member. The system is comprise a protective layer disposed adjacent to and covering a top surface of the support member.

### CROSS-REFERENCE TO RELATED APPLICATIONS

This application is a continuation of International Patent Application No. PCT/US24/58951, filed Dec. 6, 2024, which claims the benefit of U.S. Provisional Patent Application No. 63/607,162, filed on Dec. 7, 2023, U.S. Provisional Patent Application No. 63/548,598, filed on Feb. 1, 2024, U.S. Provisional Patnet Applicaation No. 63/644,264, filed on May 8, 2024, and U.S. Provisional Patent Application No. 63/644,422, file on May 8, 2024, each of which is entirely incorporated herein by reference.

### BACKGROUND

Regulating a temperature of an article of furniture (e.g., a bed) can help improve a quality of a person's activity on the furniture (e.g., sleeping on the bed). For example, fluid (e.g., liquid or gas) can be directed to flow within the article of furniture. Temperature of the fluid can be modulated to effect regulation of temperature of the article of furniture, to effect improvement of the person's quality of sleep.

A condition (e.g., temperature, posture, vibration, etc.) of an article of furniture (e.g., a bed) can be modified during a person's use of the article furniture. In some cases, the condition can be modified to improve a quality of a person's activity on the furniture (e.g., sleeping on the bed). For example, fluid (e.g., liquid or gas) can be directed to flow within the article of furniture. Temperature of the fluid can be modulated to effect regulation of temperature of the article of furniture, to effect improvement of the person's quality of sleep.

### BRIEF SUMMARY

In an aspect, the present disclosure provides a system comprising: a bed device comprising: (i) one or more sensors for detecting at least one biological signal from a user of the bed device, (ii) a temperature control unit coupled to the one or more sensors, wherein the temperature control unit is configured to adjust a temperature of a portion of the bed device, and (iii) a sensor zone positioned on a bed device, wherein the sensor zone is configured to control one or more functions of the bed device based on a contact from the user; and a processor operatively coupled to the temperature control unit and the one or more sensors.

In some embodiments of any one of the systems described herein, the sensor zone is positioned on a side surface of the bed device or a top surface of the bed device. In some embodiments of any one of the systems described herein, one or more functions comprise a temperature of the bed device, an elevation of the bed device, an alarm of the bed device, or any combination thereof. In some embodiments of any one of the systems described herein, each of the one or more functions of the bed device are controlled by a unique tap gesture of the sensor zone. In some embodiments of any one of the systems described herein, the sensor zone is configured to process at least four unique tap gestures.

In some embodiments of any one of the systems described herein, the sensor zone comprises a first sensor zone and second sensor zone. In some embodiments of any one of the systems described herein, the first sensor zone controls one or more functions of a first section of a bed device and the second sensor zone controls one or more functions of a second section of a bed device.

In some embodiments of any one of the systems described herein, the sensor zone comprises haptic feedback to the user. In some embodiments of any one of the systems described herein, the system further comprises a support member comprising a plurality of grooves, wherein the grooves are configured to house a plurality of channels configured for fluid flow.

In some embodiments of any one of the systems described herein, the support member further comprises a plurality of junctions. In some embodiments of any one of the systems described herein, a first junction of the plurality of junctions is fluidically coupled to a first end of a first channel of the plurality of channels. In some embodiments of any one of the systems described herein, the first junction is fluidically coupled to the first end of the channel of the first channel via an outlet port. In some embodiments of any one of the systems described herein, a second end of the first channel of the plurality of channels is coupled to a second junction of the plurality of junctions. In some embodiments of any one of the systems described herein, connections of the plurality of junctions with the plurality of channels provides a continuous flow of fluid through the plurality of channels. In some embodiments of any one of the systems described herein, the one or more sensors are electrically connected to one another via the plurality of junctions. In some embodiments of any one of the systems described herein, a junction of the plurality of junctions comprises a printed circuit board.

In some embodiments of any one of the systems described herein, the support member comprises a foam. In some embodiments of any one of the systems described herein, the plurality of channels protrude out of the plane of the top surface of the support member by at most about 10 cm. In some embodiments of any one of the systems described herein, the plurality of channels protrude out of the plane of the top surface of the support member by at most about 3 cm. In some embodiments of any one of the systems described herein, the plurality of grooves of the support member is separated into two or more sections of the support member. In some embodiments of any one of the systems described herein, the two or more sections of the support member comprise a first plurality of channels and a second plurality of channels. In some embodiments of any one of the systems described herein, the first plurality of channels and the second plurality of channels comprise different fluid flows. In some embodiments of any one of the systems described herein, a fluid flow of the first plurality of channels is configured to adjust a temperature of a first section of the support member to a different temperature than that of a second section of the support member. In some embodiments of any one of the systems described herein, a fluid flow of the first plurality of channels is configured to adjust a temperature of a first section of the support member to a same temperature than that of a second section of the support member.

In some embodiments of any one of the systems described herein, the fluid is a liquid. In some embodiments of any one of the systems described herein, the fluid is a gas.

In some embodiments of any one of the systems described herein, the bed device comprises a mattress. In some embodiments of any one of the systems described herein, the bed device comprises a mattress cover.

In some embodiments of any one of the systems described herein, the temperature control unit is disposed adjacent to the plurality of channels. In some embodiments of any one of the systems described herein, the temperature control unit is disposed underneath the plurality of channels. In some embodiments of any one of the systems described herein, the plurality of channels comprises a cross-sectional shape with an equal width and higher. In some embodiments of any one of the systems described herein, the plurality of channels comprises an oblong shape. In some embodiments of any one of the systems described herein, the plurality of channels comprises a thermoplastic or a thermoplastic elastomer (TPE). In some embodiments of any one of the systems described herein, the TPE comprises a polyamide thermoplastic elastomer (TPA), a polystyrene thermoplastic elastomer (TPS), a polyurethane thermoplastic elastomer (TPU), an olefinic thermoplastic elastomer (TPO), a polyester thermoplastic elastomer (TPEE), a crosslinked thermoplastic rubber (TPV), or any combination thereof.

In some embodiments of any one of the systems described herein, the one or more sensors comprise different types of sensors. In some embodiments of any one of the systems described herein, the one or more sensors comprise a capacitance sensor, a temperature sensor, a pressure sensor, a piezo sensor, or any combination thereof. In some embodiments of any one of the systems described herein, the one or more sensors comprise a piezo sensor.

In some embodiments of any one of the systems described herein, the at least one biological signal comprises a heart signal, a breathing signal, a respiration rate, a temperature, a movement, a presence of the user, or any combination thereof.

In some embodiments of any one of the systems described herein, the one or more sensors comprise a first sensor and a second sensor. In some embodiments of any one of the systems described herein, the first sensor and the second sensor are positioned at different locations of the support member. In some embodiments of any one of the systems described herein, the first sensor and the second sensor detect different biological signals. In some embodiments of any one of the systems described herein, the first sensor and the second sensor detect different biological signals from different bodily portions of the user.

In some aspects, the present disclosure provides a system comprising: a bed device comprising: (i) one or more sensors for detecting at least one biological signal from a user of the bed device, (ii) a temperature control unit coupled to the one or more sensors, wherein the temperature control unit is configured to adjust a temperature of a portion of the bed device, and (iii) a support member comprising a plurality of grooves, wherein the grooves are configured to house a plurality of channels configured for fluid flow; and a processor operatively coupled to the temperature control unit and the one or more sensors.

In some embodiments of any one of the systems described herein, the support member further comprises a plurality of junctions. In some embodiments of any one of the systems described herein, a first junction of the plurality of junctions is fluidically coupled to a first end of a first channel of the plurality of channels. In some embodiments of any one of the systems described herein, the first junction is fluidically coupled to the first end of the channel of the first channel via an outlet port. In some embodiments of any one of the systems described herein, a second end of the first channel of the plurality of channels is coupled to a second junction of the plurality of junctions. In some embodiments of any one of the systems described herein, connections of the plurality of junctions with the plurality of channels provides a continuous flow of fluid through the plurality of channels.

In some embodiments of any one of the systems described herein, the one or more sensors are electrically connected to one another via the plurality of junctions. In some embodiments of any one of the systems described herein, a junction of the plurality of junctions comprises a printed circuit board.

In some embodiments of any one of the systems described herein, the support member comprises a foam.

In some embodiments of any one of the systems described herein, the plurality of channels protrude out of the plane of the top surface of the support member by at most about 10 cm. In some embodiments of any one of the systems described herein, the plurality of channels protrude out of the plane of the top surface of the support member by at most about 3 cm.

In some embodiments of any one of the systems described herein, the plurality of grooves of the support member is separated into two or more sections of the support member. In some embodiments of any one of the systems described herein, the two or more sections of the support member comprise a first plurality of channels and a second plurality of channels. In some embodiments of any one of the systems described herein, the first plurality of channels and the second plurality of channels comprise different fluid flows. In some embodiments of any one of the systems described herein, a fluid flow of the first plurality of channels is configured to adjust a temperature of a first section of the support member to a different temperature than that of a second section of the support member. In some embodiments of any one of the systems described herein, a fluid flow of the first plurality of channels is configured to adjust a temperature of a first section of the support member to a same temperature than that of a second section of the support member.

In some embodiments of any one of the systems described herein, the fluid is a liquid. In some embodiments of any one of the systems described herein, the fluid is a gas.

In some embodiments of any one of the systems described herein, the bed device comprises a mattress. In some embodiments of any one of the systems described herein, the bed device comprises a mattress cover.

In some embodiments of any one of the systems described herein, the temperature control unit is disposed adjacent to the plurality of channels. In some embodiments of any one of the systems described herein, the temperature control unit is disposed underneath the plurality of channels. In some embodiments of any one of the systems described herein, the plurality of channels comprises a cross-sectional shape with an equal width and higher. In some embodiments of any one of the systems described herein, the plurality of channels comprises an oblong shape. In some embodiments of any one of the systems described herein, the plurality of channels comprises a thermoplastic or a thermoplastic elastomer (TPE). In some embodiments of any one of the systems described herein, the TPE comprises a polyamide thermoplastic elastomer (TPA), a polystyrene thermoplastic elastomer (TPS), a polyurethane thermoplastic elastomer (TPU), an olefinic thermoplastic elastomer (TPO), a polyester thermoplastic elastomer (TPEE), a crosslinked thermoplastic rubber (TPV), or any combination thereof.

In some embodiments of any one of the systems described herein, the one or more sensors comprise different types of sensors. In some embodiments of any one of the systems described herein, the one or more sensors comprise a capacitance sensor, a temperature sensor, a pressure sensor, a piezo sensor, or any combination thereof. In some embodiments of any one of the systems described herein, the one or more sensors comprise a piezo sensor.

In some embodiments of any one of the systems described herein, the at least one biological signal comprises a heart signal, a breathing signal, a respiration rate, a temperature, a movement, a presence of the user, or any combination thereof.

In some embodiments of any one of the systems described herein, the one or more sensors comprise a first sensor and a second sensor. In some embodiments of any one of the systems described herein, the first sensor and the second sensor are positioned at different locations of the support member. In some embodiments of any one of the systems described herein, the first sensor and the second sensor detect different biological signals. In some embodiments of any one of the systems described herein, the first sensor and the second sensor detect different biological signals from different bodily portions of the user. In some embodiments of any one of the systems described herein, the bed device further comprises a sensor zone. In some embodiments of any one of the systems described herein, the sensor zone is positioned on a side surface of the bed device, on a top surface of a bed device, or any combination thereof. In some embodiments of any one of the systems described herein, the sensor zone is configured to control the temperature of the bed device, an angular positioning of the bed device, or any combination thereof, based on a contact from the user to the sensor zone.

In some aspects, the present disclosure provides a method comprising: (a) providing the bed device comprising: (i) one or more sensors; (ii) a temperature control unit coupled to the one or more sensors, wherein the temperature control unit is configured to adjust a temperature of a portion of the bed device, and (iii) a sensor zone positioned on a bed device, wherein the sensor zone is configured to control one or more functions of the bed device based on a contact from the user; and (b) detecting, via the one or more sensors, at least one biological signal from a user of the bed device; (c) configuring a processor to receive the detected at least one biological signal; and (d) based on the detected at least one biological signal, using the temperature control unit to adjust temperature of the fluid of the plurality of channels to thereby adjust the temperature of the bed device.

In some embodiments of any one of the methods described herein, the sensor zone is positioned on a side surface of the bed device or a top surface of the bed device. In some embodiments of any one of the methods described herein, one or more functions comprise a temperature of the bed device, an elevation of the bed device, an alarm of the bed device, or any combination thereof. In some embodiments of any one of the methods described herein, each of the one or more functions of the bed device are controlled by a unique tap gesture of the sensor zone. In some embodiments of any one of the methods described herein, the sensor zone is configured to process at least four unique tap gestures. In some embodiments of any one of the methods described herein, the sensor zone comprises a first sensor zone and second sensor zone. In some embodiments of any one of the methods described herein, the first sensor zone controls one or more functions of a first section of a bed device and the second sensor zone controls one or more functions of a second section of a bed device. In some embodiments of any one of the methods described herein, the sensor zone comprises haptic feedback to the user.

In some embodiments of any one of the methods described herein, the method further comprises a support member comprising a plurality of grooves, wherein the grooves are configured to house a plurality of channels configured for fluid flow.

In some embodiments of any one of the methods described herein, the support member further comprises a plurality of junctions. In some embodiments of any one of the methods described herein, a first junction of the plurality of junctions is fluidically coupled to a first end of a first channel of the plurality of channels. In some embodiments of any one of the methods described herein, the first junction is fluidically coupled to the first end of the channel of the first channel via an outlet port. In some embodiments of any one of the methods described herein, a second end of the first channel of the plurality of channels is coupled to a second junction of the plurality of junctions. In some embodiments of any one of the methods described herein, connections of the plurality of junctions with the plurality of channels provides a continuous flow of fluid through the plurality of channels. In some embodiments of any one of the methods described herein, the one or more sensors are electrically connected to one another via the plurality of junctions. In some embodiments of any one of the methods described herein, a junction of the plurality of junctions comprises a printed circuit board.

In some embodiments of any one of the methods described herein, the support member comprises a foam. In some embodiments of any one of the methods described herein, the plurality of channels protrude out of the plane of the top surface of the support member by at most about 10 cm. In some embodiments of any one of the methods described herein, the plurality of channels protrude out of the plane of the top surface of the support member by at most about 3 cm.

In some embodiments of any one of the methods described herein, the plurality of grooves of the support member is separated into two or more sections of the support member. In some embodiments of any one of the methods described herein, the two or more sections of the support member comprise a first plurality of channels and a second plurality of channels. In some embodiments of any one of the methods described herein, the first plurality of channels and the second plurality of channels comprise different fluid flows. In some embodiments of any one of the methods described herein, a fluid flow of the first plurality of channels is configured to adjust a temperature of a first section of the support member to a different temperature than that of a second section of the support member. In some embodiments of any one of the methods described herein, a fluid flow of the first plurality of channels is configured to adjust a temperature of a first section of the support member to a same temperature than that of a second section of the support member.

In some embodiments of any one of the methods described herein, the fluid is a liquid. In some embodiments of any one of the methods described herein, the fluid is a gas.

In some embodiments of any one of the methods described herein, the bed device comprises a mattress. In some embodiments of any one of the methods described herein, the bed device comprises a mattress cover.

In some embodiments of any one of the methods described herein, the temperature control unit is disposed adjacent to the plurality of channels. In some embodiments of any one of the methods described herein, the temperature control unit is disposed underneath the plurality of channels. In some embodiments of any one of the methods described herein, the plurality of channels comprises a cross-sectional shape with an equal width and higher. In some embodiments of any one of the methods described herein, the plurality of channels comprises an oblong shape. In some embodiments of any one of the methods described herein, the plurality of channels comprises a thermoplastic or a thermoplastic elastomer (TPE). In some embodiments of any one of the methods described herein, the TPE comprises a polyamide thermoplastic elastomer (TPA), a polystyrene thermoplastic elastomer (TPS), a polyurethane thermoplastic elastomer (TPU), an olefinic thermoplastic elastomer (TPO), a polyester thermoplastic elastomer (TPEE), a crosslinked thermoplastic rubber (TPV), or any combination thereof.

In some embodiments of any one of the methods described herein, the one or more sensors comprise different types of sensors. In some embodiments of any one of the methods described herein, the one or more sensors comprise a capacitance sensor, a temperature sensor, a pressure sensor, a piezo sensor, or any combination thereof. In some embodiments of any one of the methods described herein, the one or more sensors comprise a piezo sensor.

In some embodiments of any one of the methods described herein, the at least one biological signal comprises a heart signal, a breathing signal, a respiration rate, a temperature, a movement, a presence of the user, or any combination thereof.

In some embodiments of any one of the methods described herein, the one or more sensors comprise a first sensor and a second sensor. In some embodiments of any one of the methods described herein, the first sensor and the second sensor are positioned at different locations of the support member. In some embodiments of any one of the methods described herein, the first sensor and the second sensor detect different biological signals. In some embodiments of any one of the methods described herein, the first sensor and the second sensor detect different biological signals from different bodily portions of the user.

In some aspects, the present disclosure provides a method for adjusting a temperature of a bed device, comprising: (a) providing the bed device comprising: (i) one or more sensors; (ii) a temperature control unit coupled to the one or more sensors, wherein the temperature control unit is configured to adjust a temperature of a portion of the bed device, and (iii) a support member comprising a plurality of grooves, wherein the grooves are configured to house a plurality of channels configured for fluid flow; and (b) detecting, via the one or more sensors, at least one biological signal from a user of the bed device; (c) configuring a processor to receive the detected at least one biological signal; and (d) based on the detected at least one biological signal, using the temperature control unit to adjust temperature of the fluid of the plurality of channels to thereby adjust the temperature of the bed device.

In some embodiments of any one of the methods described herein, the support member further comprises a plurality of junctions. In some embodiments of any one of the methods described herein, a first junction of the plurality of junctions is fluidically coupled to a first end of a first channel of the plurality of channels. In some embodiments of any one of the methods described herein, the first junction is fluidically coupled to the first end of the channel of the first channel via an outlet port. In some embodiments of any one of the methods described herein, a second end of the first channel of the plurality of channels is coupled to a second junction of the plurality of junctions. In some embodiments of any one of the methods described herein, connections of the plurality of junctions with the plurality of channels provides a continuous flow of fluid through the plurality of channels. In some embodiments of any one of the methods described herein, the one or more sensors are electrically connected to one another via the plurality of junctions. In some embodiments of any one of the methods described herein, a junction of the plurality of junctions comprises a printed circuit board.

In some embodiments of any one of the methods described herein, the support member comprises a foam.

In some embodiments of any one of the methods described herein, the plurality of channels protrude out of the plane of the top surface of the support member by at most about 10 cm. In some embodiments of any one of the methods described herein, the plurality of channels protrude out of the plane of the top surface of the support member by at most about 3 cm.

In some embodiments of any one of the methods described herein, the plurality of grooves of the support member is separated into two or more sections of the support member. In some embodiments of any one of the methods described herein, the two or more sections of the support member comprise a first plurality of channels and a second plurality of channels. In some embodiments of any one of the methods described herein, the first plurality of channels and the second plurality of channels comprise different fluid flows. In some embodiments of any one of the methods described herein, a fluid flow of the first plurality of channels is configured to adjust a temperature of a first section of the support member to a different temperature than that of a second section of the support member. In some embodiments of any one of the methods described herein, a fluid flow of the first plurality of channels is configured to adjust a temperature of a first section of the support member to a same temperature than that of a second section of the support member.

In some embodiments of any one of the methods described herein, the fluid is a liquid. In some embodiments of any one of the methods described herein, the fluid is a gas.

In some embodiments of any one of the methods described herein, the bed device comprises a mattress. In some embodiments of any one of the methods described herein, the bed device comprises a mattress cover.

In some embodiments of any one of the methods described herein, the temperature control unit is disposed adjacent to the plurality of channels. In some embodiments of any one of the methods described herein, the temperature control unit is disposed underneath the plurality of channels. In some embodiments of any one of the methods described herein, the plurality of channels comprises a cross-sectional shape with an equal width and higher. In some embodiments of any one of the methods described herein, the plurality of channels comprises an oblong shape. In some embodiments of any one of the methods described herein, the plurality of channels comprises a thermoplastic or a thermoplastic elastomer (TPE). In some embodiments of any one of the methods described herein, the TPE comprises a polyamide thermoplastic elastomer (TPA), a polystyrene thermoplastic elastomer (TPS), a polyurethane thermoplastic elastomer (TPU), an olefinic thermoplastic elastomer (TPO), a polyester thermoplastic elastomer (TPEE), a crosslinked thermoplastic rubber (TPV), or any combination thereof.

In some embodiments of any one of the methods described herein, the one or more sensors comprise different types of sensors. In some embodiments of any one of the methods described herein, the one or more sensors comprise a capacitance sensor, a temperature sensor, a pressure sensor, a piezo sensor, or any combination thereof. In some embodiments of any one of the methods described herein, the one or more sensors comprise a piezo sensor.

In some embodiments of any one of the methods described herein, the at least one biological signal comprises a heart signal, a breathing signal, a respiration rate, a temperature, a movement, a presence of the user, or any combination thereof.

In some embodiments of any one of the methods described herein, the one or more sensors comprise a first sensor and a second sensor. In some embodiments of any one of the methods described herein, the first sensor and the second sensor are positioned at different locations of the support member. In some embodiments of any one of the methods described herein, the first sensor and the second sensor detect different biological signals. In some embodiments of any one of the methods described herein, the first sensor and the second sensor detect different biological signals from different bodily portions of the user. In some embodiments of any one of the methods described herein, the bed device further comprises a sensor zone. In some embodiments of any one of the methods described herein, the sensor zone is positioned on a side surface of the bed device, on a top surface of a bed device, or any combination thereof. In some embodiments of any one of the methods described herein, the sensor zone is configured to control the temperature of the bed device, an angular positioning of the bed device, or any combination thereof, based on a contact from the user to the sensor zone.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and the disclosure will be obtained by reference to the following detailed description that sets forth illustrative embodiments, in which the principles of the present disclosure are utilized, and the accompanying drawings (also “Figure” and “FIG.” herein), of which:

FIG. 1 shows an isometric view of an example system comprising a plurality of channels and a support member comprising a plurality of grooves.

FIG. 2 schematically illustrates a cross-sectional view of an example support member comprising a plurality of grooves.

FIG. 3 schematically illustrates a cross-sectional view of an example system comprising a plurality of channels disposed within a plurality of grooves of a support member.

FIG. 4 schematically illustrates an isometric view of an example system comprising a support member and a fluidic system.

FIG. 5 schematically illustrates a cross-sectional view of an example system comprising a support member and a fluidic system.

FIG. 6 schematically illustrates a side view of an example system comprising a support member having varying thickness and/or stiffness along the length of the support member.

FIG. 7 schematically illustrates an example of a sensor unit.

FIG. 8 schematically illustrates a top view of an example system comprising sensor units disposed over a support member.

FIG. 9 schematically illustrates a rotary convolution cut process.

FIG. 10 schematically illustrates a non-rotary convolution cut process.

FIG. 11 schematically illustrates two molds for a non-rotary convolution cut process.

FIG. 12 shows a portion of an example article of furniture comprising a protective layer covering a sensor, fluid channels, and a support member.

FIG. 13 schematically illustrates an example of an article of furniture.

FIG. 14 shows a perspective view of an article of furniture comprising a textured surface.

FIG. 15 schematically illustrates a cross-section of an example sensor zone of an article of furniture.

FIG. 16 shows a portion of an example sensor zone of an article of furniture.

FIG. 17 shows a computer system that is programmed or otherwise configured to implement methods provided herein.

FIG. 18 shows an example of the tap control feature of the bed device. The sensor control zone is on the side of bed device and can regulate temperature, base election, alarm, or any combination thereof.

FIG. 19 shows an exemplary schematic of the sensor zone on the side of a bed device. The sensor zone is illuminated on the side of the bed device and can show the user where to tap to control features of the bed device.

FIG. 20 shows examples of tap gestures used with the tap control feature of the sensor zone of the bed device.

### DETAILED DESCRIPTION

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure herein can be described in the context of a single embodiment, the features can also be provided separately or in any suitable combination. Conversely, although the present disclosure herein can be described herein in the context of separate embodiments for clarity, the inventions of the present disclosure herein can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting. In this application, the use of the singular includes the plural unless specifically stated otherwise. It must be noted that, as used in the specification, the singular forms “a,”“an”and “the”include plural referents unless the context clearly dictates otherwise.

In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most,” “up to,” “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “furniture,” “article of furniture,” or “piece of furniture,” as used interchangeably herein, generally refer to a bed, a pillow, crib, bassinet, chair, seat, loveseat, sofa, couch, head rest, stool, ottoman, bench, or any panel intended to be covered with a fabric. The article of furniture can be intended for use in a home, an office, a medical facility (e.g., a hospital), or on a vehicle of transportation such as a car, truck, boat, bus, train or the like. The article of furniture can be intended for use for at least one person (and/or at least one animal, such as a pet). The article of furniture can be intended for use for at least about 1, 2, 3, 4, 5, 6, 7, 8, 9, 10, or more persons. The article of furniture can be intended for use for at most about 10, 9, 8, 7, 6, 5, 4, 3, 2, or 1 person. In an example, the article of furniture may be a bed, and the bed may comprise a plurality of sizes comprising single, single extra-long, double, queen, king, super king, etc. In another example, the article of furniture may be an infant warmer (i.e., a babytherm) to provide heat at one or more temperatures to an infant.

The terms “bed” or “bed device,” as used interchangeably herein, may be an article of furniture used for sleep or rest. The bed may comprise a mattress, a mattress pad, a pillow, and/or a covering thereof (e.g., a blanket). One or more users may sleep or rest on and/or adjacent to a surface of the bed device. The surface may be a top surface of the bed device. The top surface of the bed device may be flat or textured. The bed device may be a mattress. The bed device may be a mattress pad that covers at least a portion of a surface of a mattress or at least a surface of the mattress. The bed device may be a pillow. Alternatively or in addition to, the user(s) may sleep under a surface of the bed device. The surface may be one or more surfaces of a covering, such as, for example, a blanket. The blanket may be disposed on top of at least a part of the user(s). The bed device may be the blanket.

### Systems and Methods for Temperature Regulation of a Bed Device

The present disclosure provides systems for regulating a temperature of an article of furniture, and methods of use thereof.

Various aspects of the present disclosure provide systems and methods for providing or regulating a setting of an article of furniture. Non-limiting examples of a setting of an article of furniture can include temperature, shape, movement (e.g., vibration), etc. The article of furniture can be, for example, a bed device, chair, or sofa. Thus, any disclosures directed to one example of an article of furniture (e.g., a bed device) may be interchangeable with another example of an article of furniture (e.g., chair, sofa, etc.).

Various aspects of the systems and methods provided herein can be utilized to tailor a setting of the article of furniture (e.g., temperature of a bed device) based on one or more parameters specific for a user of the article of furniture. Non-limiting examples of such parameter(s) can include, age, biological sex, geolocation, time (e.g., with respect to the day, with respect to the week, with respect to the month, with respect to the year, with respect to the season) of use of the article of furniture, sleep habits or patterns (e.g., average bedtime), health condition or state (e.g., healthy, a disease type, pregnancy, menstruation cycle, menopause, hot flashes, night sweats, etc.). The setting of the article of furniture can be tailored prior to, during, and/or subsequent to the user's use of the article of furniture. For example, a temperature of a bed device can be adjusted, via the system and methods provided herein, prior to the user's getting into the bed device, prior to the user's falling asleep on the bed device, during the user's sleep on the bed device, prior to the user's waking up while sleeping on the bed device, during the user's waking up on the bed device, and/or subsequent to the user's use of the bed device.

The systems and methods of the present disclosure can be utilized for regulating temperature of a surface. In some embodiments, the surface is a bed device. In some embodiments, the bed device changes temperature. In some embodiments, the bed device changes temperature while a subject is on the surface of the bed device. In some embodiments, the bed device is operatively coupled to one or more sensors. The one or more sensors can be part of the bed device, part of a room or space comprising the bed device there within, and/or a part of a wearable device that the user of the bed device is wearing. The sensor(s) can be configured to measure one or more biological signals comprising heart signal, breathing signal, respiration rate, temperature, movement of the user, presence of the user, or any combination thereof. Non-limiting examples of a sensor can include a capacitance sensor (e.g., for detecting presence), a piezo sensor (e.g., for measuring a biological signal such as heart signal, breathing signal, respiration signal, etc.), a temperature sensor (e.g., for measuring temperature of the user, or that of the bed), pressure sensor, optical sensor, humidity sensors, etc.

In some embodiments, the bed device further comprises a processor. In some embodiments, the processor analyzes the data collected by the one or more sensors. In some embodiments, the bed device further comprises a temperature control unit. In some embodiments, the temperature control unit is operatively connected to the processor. In some embodiments, the processor can provide a control signal to the temperature control unit. In some embodiments, the control signal can be an instruction for the temperature control unit to change a temperature of the article of furniture. In some embodiments, the control signal can be an instruction for the temperature control unit to change a temperature of a portion of the article of furniture (e.g., bed device). In some embodiments, the temperature control unit is capable of directing a fluid that is temperature regulated on at least a portion of the bed device. In some embodiments, the temperature control unit is capable of directing a fluid that is temperature regulated adjacent to the at least the portion of the bed device. In some cases, the fluid is liquid. In some cases, the fluid is a gas. In some cases, the gas is air. In some cases, the temperature control unit is located within the bed device. In some cases, the temperature control unit is located separate from the bed device.

A temperature of the article of furniture (e.g., the bed device, such as the mattress, the mattress pad, pillow, or the blanket) may be controlled (e.g., increasing, decreasing, or maintaining the temperature of the bed). A temperature of at least a portion of the article of furniture may be controlled. The temperature of the article of furniture may be adjustable or maintained prior to, during, or subsequent to a use (e.g., sleeping or resting for a period of time) by the user(s). In an example, the bed may be pre-warmed (e.g., automatically or per user preference) prior to the use by the user(s). In some cases, temperatures or two or more portions of the article of furniture (e.g., the bed) may be controlled separately or in sync.

The article of furniture (e.g., the bed) may use one or more sensors and/or one or more computer systems to detect sensing data (e.g., one or more biological signals) associated with the user. For example, the sensing data can be utilized to estimate or determine a condition of state of the user prior to, during, or subsequent to using the article of furniture (e.g., determine sleep phase, sleep pattern, disease, disorder, etc. of the user). The sensor(s) may or may not be a part of the article of furniture. The sensor(s) may be part of the article of furniture. The sensor(s) may be a part of a space (e.g., room) surrounding the article of furniture. The sensor(s) may be worn by the user(s). Non-limiting examples of a sensor can include a capacitance sensor, a temperature sensor, a pressure sensor, a piezoelectric sensor (e.g., a piezo sensor), or any combination thereof. The sensing data can be utilized (e.g., analyzed), at least in part, to determine how to regulate temperature of the article of furniture prior to, during, and subsequent to the user's use of the article of furniture. In some cases, the sensor(s) may be used to detect a property (e.g., temperature, movement, etc.) of the article of furniture or such property of an environment surrounding the article of furniture.

In some embodiments, the sensor(s) of the article of furniture can be disposed within a portion of the article of furniture that corresponds to a target bodily portion of the user, such as head, arms, legs, torso, upper body, lower body, etc.

A disorder of a user can be a sleep disorder. Non-limiting examples of the sleep disorder may include dyssomnias, such as insomnia, primary hypersomnia (e.g., narcolepsy, idiopathic hypersomnia, recurrent hypersomnia, posttraumatic hypersomnia, menstrual-related hypersomnia), sleep disordered breathing (e.g., sleep apnea, snoring, upper airway resistance syndrome), circadian rhythm sleep disorders (e.g., delayed sleep phase disorder, advanced sleep phase disorder, non-24-hour sleep-wake disorder), parasomnias (e.g., bedwetting, bruxism, catathrenia, exploding head syndrome, sleep terror, REM sleep behavior disorder, sleep talking), jet lag, restless legs syndrome, etc.

In some embodiments, the article of furniture can be a bed (e.g., a mattress or a mattress cover), and the temperature of the bed can be controlled (e.g., based at least in part on the sensing data) to assist the user(s) to fall asleep, assist the user to wake up from sleeping, promote enhanced sleep quality, treat or ameliorate the disorder of the user while sleeping, etc.

The terms “biological signal” and “bio signal” can be used interchangeably. Examples of the biological signal can include a heart signal (e.g., heart rate or sound), a respiration (breathing) signal (e.g., respiration rate or sound), a motion, a temperature, a movement, perspiration, sound, neural activity, etc. The article of furniture (e.g., the bed) may be capable of detecting one or more biological signals of the user(s). The article of furniture may be capable of adjusting a property of the article of furniture (e.g., temperature or movement of the article of furniture, such as vibration, geometric configuration, etc.) to control (e.g., increase, decrease, or maintain) the biological signal(s) of the user(s) of the article of furniture.

The term “module” refers broadly to software, hardware, or firmware components (or any combination thereof). Modules are typically functional components that can generate useful data or another output using specified input(s). A module may or may not be self-contained. An application program (also called an “application”) may include one or more modules, or a module may include one or more application programs.

The term “on top of” can mean that the two objects, where the first object is “on top of” the second object, can be rotated so that the first object is above the second object relative to the ground. The two objects can be in direct or indirect contact, or may not be in contact at all.

The term the term “channel”, as used herein, can generally means any structure that is capable of being a medium for allowing a fluid to flow, e.g., between at least two locations. Non-limiting examples of a channel can include a pipe, a hose, a tube, a duct, etc. A channel can be transparent, semi-transparent, or non-transparent. A channel can be flexible or substantially rigid. A channel can be made of a polymer (e.g., plastic) or ceramic material (e.g., glass).

In some embodiments, the system can comprise one or more channels for fluid (e.g., liquid or gas) to flow along and within the one or more channels. The one or more channels can be applied to or disposed in an article of furniture (e.g., a bed device or a portion thereof, such as a mattress, a mattress cover, a pillow, or a pillow cover). Temperature of the fluid can be modulated (e.g., subsequent to, during, or prior to flowing within the one or more channels) to effect regulation of temperature of the article of furniture.

In some embodiments, the one or more channels can be a single channel. The one or more channels can be a plurality of channels. The plurality of channels may or may not be in fluid communication with one another The plurality of channels can be coupled to a common junction (e.g., a single junction). The common junction can comprise a plurality of ports to physically and fluidically couple to the plurality of channels. The common junction can comprise at least one additional port to physically and fluidically couple to a source of the fluid (e.g., a common source of the fluid). In some embodiments, the plurality of channels may not and need not be in fluid communication with one another, except for at the common junction. Even when a first channel and a second channel (which are not directly coupled to the source of the fluid) are both connected to the common junction, fluid from the first channel may not directly flow from the first channel, through the common junction, and into the second channel.

In some embodiments, two different channels may be in fluid communication via at most two common junctions or at most one common junction.

In some embodiments, the plurality of channels may not be a watermat (e.g., a sheet of interconnected rows and columns are fluid passages).

In some embodiments, the system can comprise a support member for holding the one or more channels. The one or more channels be disposed on or adjacent to a top surface of the support member. The support member can comprise one or more grooves (or trenches) on the top surface, such that at least a portion of a channel can be disposed within a groove. The depth of a groove of the support member may be shorter than the thickness of the support member, such that a groove does not go all the way through the support member (e.g., the groove does not form an opening through the thickness of the support member). Accordingly, the groove may not be visible from a bottom surface of the support member, which bottom surface is opposite the top surface. The depth of the groove may be less than, equal to, or greater than a cross-sectional dimension (e.g., height or diameter) of a channel. For example, the depth of the groove can be greater than or equal to the cross-sectional dimension of the channel, such that when the channel is inserted into the groove, the top portion of the channel does not protrude out of the top surface of the support member. The support member can comprise a plurality of grooves for holding a plurality of channels.

In some embodiments, a width of at least a portion of the groove as provided herein (e.g., a dimension substantially perpendicular to the length of the channel) can be substantially the same as a width of the channel (e.g., an external dimension such as diameter that is substantially perpendicular to the length of the channel). Alternatively, the width of the at least the portion of the groove (e.g., bottom portion, middle portion, top portion, etc.) can be different (e.g., greater than or smaller than) as compared to the width of the channel by at least or at most about 1%, at least or at most about 2%, at least or at most about 3%, at least or at most about 4%, at least or at most about 5%, at least or at most about 6%, at least or at most about 7%, at least or at most about 8%, at least or at most about 9%, at least or at most about 10%, at least or at most about 15%, at least or at most about 20%, at least or at most about 25%, at least or at most about 30%, at least or at most about 40%, at least or at most about 50%, at least or at most about 60%, at least or at most about 70%, or at least or at most about 80%.

In some embodiments, the width of the groove can be substantially uniform along the vertical axis of the groove. In some embodiments, the width of the groove can be non-uniform along the vertical axis of the groove. The width of the groove can change (e.g., increase, decrease, fluctuate in a wave or zig-zag pattern, etc.) along the vertical direction of the groove.

In some embodiments, a surface of the groove or a surface of the channel can be substantially flat. In some embodiments, the surface of the groove or the surface of the channel can be textured.

In some embodiments, the support member can be flexible or substantially rigid.

In some embodiments, the support member can be a foam. The foam can comprise a polymeric foam. Non-limiting examples of a polymeric foam can include ethylene-vinyl acetate (EVA) foam, low-density polyethylene (LDPE) foam, nitrile rubber (NBR) foam, polychloroprene foam, polyimide foam, polypropylene foam, polystyrene foam, polyurethane foam, polyurea foam, polyethylene foam, polyvinyl chloride foam, and silicone foam. For example, the foam can be a polyurethane foam.

The support member may comprise a synthetic fiberfill material. The term “fiberfill material” as used herein can refer to a material formed of multiple staple fibers (e.g., synthetic fibers) configured to have insulating and/or cushioning properties (e.g., lofty properties) and may comprise, for example, polyester fiberfill, polyfill, and the like. In some embodiments, the staple fibers may be unbonded or unentangled, or bonded or entangled. The support member may be a polyester fiberfill (e.g., a polyfill).

In some embodiments, the one or more grooves may or may not be straight along the length of the groove(s). For example, a groove may have one or more curvatures (e.g., a non-straight portion) along a length of the groove, and the channel (e.g., a tube) can be flexible enough to bend into a shape of the curvature of the groove when and once inserted into the groove.

In some embodiments, the support member can be a foam (e.g., a sheet of foam), and the channels can be elastomeric tubes laid into grooves that are cut into (but not all the way through) the foam. Comfort level of a user when using the article of furniture comprising the system of the present disclosure (e.g., when being on top of a complex comprising the support member and the channels disposed into the grooves of the support member) can be adjusted through selection of tubes and foam with desired or target properties (e.g., softness, hardness, elasticity, etc.). Durability of the complex can be adjusted through selection of selection of tubes and foam with desired or target materials or dimensions (e.g., thickness). Thermal performance of the complex (e.g., the capability to retain thermal energy or facilitate transfer of thermal energy between the fluid in the channel and at least a portion of the article of furniture disposed adjacent to the channel) can be adjusted through selection of tubes and foam with desired or target dimensions (e.g., thickness, size), material, pattern (e.g., non-straight patters of the tubes when inserted into the grooves of the support member foam), depth of the grooves, density of the grooves on the support member, etc.

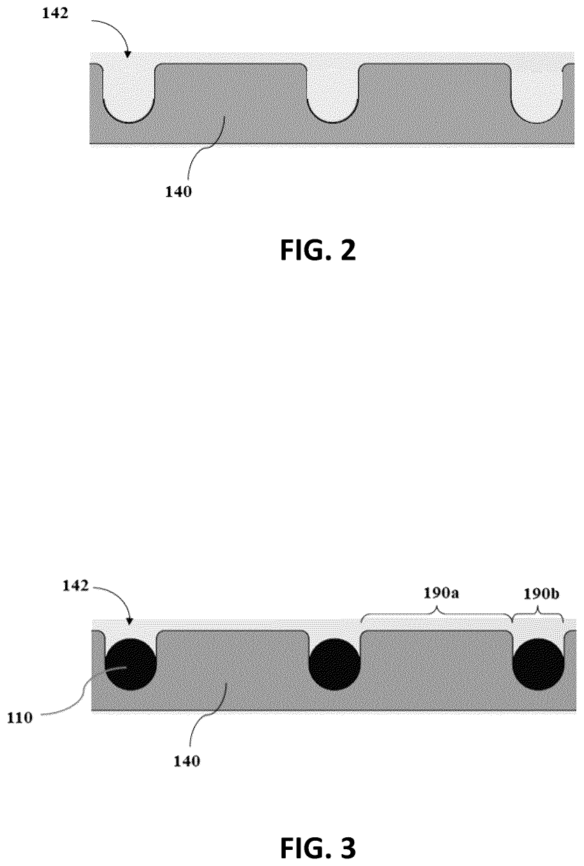

In some embodiments, the channels of the support member can have a cross-sectional shape (e.g., perpendicular to the length of the grooves) that is circular, triangular, square, rectangular, pentagonal, hexagonal, or any partial shape or combination of shapes thereof. In some embodiments, the grooves of the support member can have a cross-sectional shape (e.g., perpendicular to the length of the grooves) that is circular, triangular, square, rectangular, pentagonal, hexagonal, or any partial shape or combination of shapes thereof. In some embodiments, a portion of the groove (e.g., a bottom portion of the groove) can have a cross-sectional shape that is at least partially (e.g., substantially) inverse to a cross-sectional shape of a portion of the channel that is adjacent to or in contact with the portion of the groove. The cross-sectional shape of the groove and the cross-sectional shape of the channel can be inverted shapes to one another (e.g., a positive image shape and a negative image shape), as demonstrated in FIG. 2 and FIG. 3.

In some embodiments, the cross-sectional shape (e.g., perpendicular to the length of the grooves) of the channel of the support member can be a shape that has substantially the same width and height (e.g., a square, a circle, etc.). The article of furniture comprising such shaped channels can provide sufficient thermal exchange between the fluid flowing through the channel and an object (or a subject, such as the user) disposed on or adjacent to such fluid channel.

In some embodiments, the cross-sectional shape of the channel can be oblong, e.g., a shape that is defined by two orthogonal dimension that are different (e.g., wider than it is tall, or taller than it is wide). Non-limiting examples of the oblong shapes can include a rectangle with sharp corners, a rounded rectangle with one or more (e.g., at least or at most about 1, 2, 3, or 4) rounded corners, a racetrack shape, or an oval. One of the two orthogonal dimensions of the oblong shape can be greater than the other of the two orthogonal dimensions of the oblong shape by at least or at most about 1%, at least or at most about 2%, at least or at most about 5%, at least or at most about 10%, at least or at most about 15%, at least or at most about 20%, at least or at most about 25%, at least or at most about 30%, at least or at most about 40%, at least or at most about 50%, at least or at most about 60%, at least or at most about 70%, at least or at most about 80%, at least or at most about 90%, at least or at most about 100%, at least or at most about 120%, at least or at most about 150%, at least or at most about 200%, at least or at most about 250%, at least or at most about 300%, at least or at most about 400%, or at least or at most about 500%.

In some embodiments, the cross-sectional shape of the channel can have a width (e.g., as defined along an axis that is substantially parallel to the planar dimension of the support member) and a height (e.g., as defined along an additional axis that is substantially perpendicular to the planar dimension of the support member), and the width can be wider than the height, to increase or maximize the surface area that is exposed to the temperature regulated fluid that is flowing along the channel. For example, given substantially equal cross-sectional area, a fluid channel with an oblong cross-sectional area can have a portion of the top surface that is substantially flat for thermal exchange between the fluid flowing through the channel and an object (or a subject, such as the user) disposed on or adjacent to such fluid channel.

In some embodiments, the one or more channels and/or grooves of the support member may be arranged in any pattern. The one or more channels and/or grooves may intersect. In some embodiments, the one or more channels and/or grooves may not intersect. For example, one or more channels and/or grooves may be parallel to one another. In some embodiments, one or more channels and/or grooves may be perpendicular to one another. A channel and/or groove may comprise an angle between another channel and/or groove. For example, an angle between a channel and/or groove of the support member and another channel and/or groove may be at least about 1 degree (1°), at least about 2°, at least about 3°, at least about 4°, at least about 5°, at least about 10°, at least about 15°, at least about 20°, at least about 25°, at least about 30°, at least about 35°, at least about 40°, at least about 45°, at least about 50°, at least about 60°, at least about 70°, at least about 80°, at least about 90°, at least about 100°, at least about 120°, at least about 140°, at least about 160°, at least about 180°, at least about 200°, or greater than about 200°. In some embodiments, an angle between a channel and/or groove of the support member and another channel and/or groove may be at most about 200°, at most about 180°, at most about 160°, at most about 140°, at most about 120°, at most about 100°, at most about 90°, at most about 80°, at most about 70°, at most about 60°, at most about 50°, at most about 45°, at most about 40°, at most about 35°, at most about 30°, at most about 25°, at most about 20°, at most about 15°, at most about 10°, at most about 5°, at most about 4°, at most about 3°, at most about 2°, at most about 1°, or less than about 1°. The channels and/or grooves may start from a central point and radiate outward or spiral around, much like the spokes of a wheel or a snail shell pattern. In some embodiments, one or more channels and/or grooves may be arranged around the perimeter of the support member, with additional channels and/or grooves branching inward.

In some embodiments, as schematically illustrated in FIG. 3, a surface of the support member (e.g., a top surface facing towards a user of the article of furniture during use) can comprise a first portion 190a that does not comprise the channels 110 (or the grooves 142) and a second portion 190b comprising the channels 110 (or the grooves 142). In some cases, the area (e.g., projected area) of the second portion 190b relative to the entire area of the surface of the support member (e.g., a projected area of both the first portion 190a and the second portion 190b) can be at least or at most about 5%, at least or at most about 10%, at least or at most about 15%, at least or at most about 20%, at least or at most about 25%, at least or at most about 30%, at least or at most about 35%, at least or at most about 40%, at least or at most about 45%, at least or at most about 50%, at least or at most about 55%, at least or at most about 60%, at least or at most about 65%, at least or at most about 70%, at least or at most about 75%, at least or at most about 80%, at least or at most about 85%, at least or at most about 90%, at least or at most about 95%, or at least or at most about 99%. In some embodiments, the entire area of the surface of the support member as provided herein can be at least or at most about 15 square feet (sq. ft.), at least or at most about 20 sq. ft, at least or at most about 25 sq. ft, at least or at most about 30 sq. ft, at least or at most about 35 sq. ft, at least or at most about 40 sq. ft, at least or at most about 45 sq. ft, at least or at most about 50 sq. ft, or at least or at most about 60 sq. ft. In some embodiments, the area of the second portion 190b relative to the entire area of the surface of the support member can be associated with (or can be relative to) density of the fluid flowing across or within the support member of the article of furniture at any given point.

In some embodiments, the channel can be a tube made of a polymer, such as a thermoplastic or a thermoplastic elastomer (TPE). Non-limiting examples of a thermoplastic include polypropylene, polyethylene, polystyrene, polyurethane, polymethyl methacrylate, acrylonitrile butadiene styrene, polyamide, polylactic acid, polycarbonate, polyoxymethylene, polyester, polyketone, polyacrylate, polyether, polyvinyl ester, polyvinyl chloride, polyfluoroalkyl substance, variants thereof, and combinations thereof. Non-limiting examples of a thermoplastic elastomer can include a polyamide thermoplastic elastomer (TPA), a polystyrene thermoplastic elastomer (TPS), a polyurethane thermoplastic elastomer (TPU), an olefinic thermoplastic elastomer (TPO), a polyester thermoplastic elastomer (TPEE), a crosslinked thermoplastic rubber (TPV), and other thermoplastic elastomers (TPZ).

In some embodiments, the channel can be a tube made of a TPS. The TPS can be a copolymer comprising a first repeating unit comprising styrene and a second repeating unit lacking styrene, e.g., styrene-butadiene copolymer or styrene-isoprene copolymer. In some cases, the TPS can be a styrene block copolymer (SBC) having the poly(styrene-b-elastomer-b-styrene) structure, in which chains or phases comprising each of the repeating units are chemically bonded by block copolymerization. In some cases, the TPS can be SBC having the poly(styrene-b-butadiene-b-styrene) (SBS) structure having the butadiene-based soft phase. In some cases, the TPS can be SBC having the poly(styrene-b-ethylene/butylene-b-styrene) (SEBS) structure having the ethylene/butylene soft phase. The ethylene/butylene soft phase can be a copolymer of ethylene and butylene (e.g., random copolymer, block copolymer, etc.).

In some embodiments, a channel (e.g., a tube) can be resistant to damage (e.g., puncture, tear, etc.) from use or external forces. For example, the resistance to damage can be assessed in accordance with any testing procedures provided by the American Society for Testing and Materials (ASTM), such as, but not limited to, the Standard Test Method for Puncture-Propagation Tear Resistance of Plastic Film and Thin Sheeting (D2582), the Standard Test Method for Protective Clothing Material Resistance to Puncture (F1342), and/or the Standard Test Method for Slow Rate Penetration Resistance of Flexible Barrier Films and Laminates (F1306).

In some embodiments, a stiffness of a channel (e.g., a tube) can be soft and/or elastic to promote comfort to the user during use of the system. The stiffness of the channel can be characterized to be at least or at most about 0, at least or at most about 5, at least or at most about 10, at least or at most about 15, at least or at most about 20, at least or at most about 25, at least or at most about 30, at least or at most about 35, at least or at most about 40, at least or at most about 45, at least or at most about 50, at least or at most about 55, at least or at most about 60, at least or at most about 65, or at least or at most about 70, in accordance with the Shore A hardness scale.

In some embodiments, a channel (e.g., a tube) can exhibit chemical resistance, e.g., resistance against oils or chemicals (e.g., chemicals in liquids) that are spilled over or adjacent to the channel or the support member. For example, the chemical resistance can be assessed in accordance with the standards of Evaluating the Resistance of Plastics to Chemical Reagents by ASTM (D543).

In some embodiments, a channel (e.g., a tube) can exhibit resistance to water permeability, e.g., resistance to certain level of water evaporation from the channel and towards outside the channel.

In some embodiments, the channel can comprise a single layer. In some embodiments, the channel can comprise a plurality of layers (e.g., a first tubing disposed within a second tubing) made of the same material (e.g., same polymers) or different materials (e.g., different polymers. Having the plurality of layers (e.g., at least or at most about 2 layers, at least or at most about 3 layers, at least or at most about 4 layers, at least or at most about 5 layers, etc.) of different materials can allow the resulting channel to exhibit properties of the different materials, thereby to enhance function of the channel (e.g., a hybrid channel). The hybrid channels can be manufactured by various methods, e.g., via co-extrusion of different materials into target thicknesses for each layer of the hybrid channel. The hybrid channel can comprise two channel layers having the same thickness or different thicknesses. The two channel layers can comprise an outer channel layer and an inner channel layer. The thickness of the outer channel layer can be thicker than the thickness of the inner channel layer (or vice versa) by at least or at most about 1%, at least or at most about 5%, at least or at most about 10%, at least or at most about 15%, at least or at most about 20%, at least or at most about 30%, at least or at most about 40%, at least or at most about 50%, at least or at most about 60%, at least or at most about 70%, at least or at most about 80%, at least or at most about 90%, at least or at most about 95%, or at least or at most about 99%.

In some embodiments, the channel may be coupled to the groove of the support member via a coupling agent, such as an adhesive (e.g., glue). In some embodiments, the channel may be disposed on or within the groove in absence of any adhesive material between the channel and the groove.

In some embodiments, the plurality of channels can be in fluid communication with one another via at least one fluid junction. The at least one fluid junction can comprise an entry fluid junction that receives the fluid (e.g., temperature controlled fluid from a temperature control unit configured to adjust temperature of the fluid) and directs the fluid into the plurality of channels. The entry fluid junction can comprise a housing comprising (1) at least one entry orifice (or opening) that is connected to a first end of a channel, which channel has a second end that is connected to a source of the fluid, such as the temperature control unit and (2) a plurality of exit orifices, wherein each exit orifice is connected to a first end of one of the plurality of channels. The at least one fluid junction can comprise an exit fluid junction that receives the fluid from the plurality of channels, and direct the received fluid towards (i) the entry fluid junction and/or (ii) the temperature control unit. The exit fluid junction can comprise a housing comprising (1) a plurality of entry orifices, wherein each entry orifice is connected to a second end (different from the first end) of the plurality of channels for receiving fluid from each channel and (2) an exit orifice connected to (i) the entry fluid junction and/or (ii) the temperature control unit.

In some embodiments, the collection of the plurality of channels, the at least one fluid junction, and the temperature control unit can be referred to as a temperature control device.

In some embodiments, the temperature control unit is not a part of the article of furniture but is configured to be adjacent to the article of furniture. In some cases, the temperature control unit can comprise a housing that is separate from the article of furniture, and the housing can comprise (or contain) (i) a source (e.g., a container) of the fluid and (ii) at least one heating and/or cooling mechanism. The at least one heating and/or cooling mechanism can be configured to regulate -temperature of the fluid (1) when the fluid is contained in the source or (2) during or subsequent to when the fluid is drawn out of the source (e.g., to minimize the amount of fluid that needs to be temperature-adjusted). For example, thermoelectric temperature regulation can be implemented using an electric-based system (e.g., by a thermoelectric engine). The thermoelectric engine can be configured to convert electrical energy into a heat flux (or a temperature difference), or convert the heat flux into electrical energy. The thermoelectric engine can be a solid-state device, such as a Peltier device.

In some embodiments, a system for regulating temperatures may comprise one or more portions of an article of furniture (e.g., a bed, mattress, or mattress pad). The system may comprise a reservoir configured to contain a fluid (e.g., water). The reservoir may comprise a container (e.g., a removable or non-removable container) configured to contain a fluid. In some embodiments, neither the reservoir nor the container may be configured to modulate the temperature of the fluid that is contained in the container. In some embodiments, the reservoir and/or the container may be configured to modulate the temperature of the fluid that is contained in the container. The system may comprise fluid loops that are in fluid communication with the container of the reservoir. The fluid loops may or may not be in fluid communication with each other. The reservoir may serve as a common reservoir for the fluid loops of the system. A fluid loop may comprise (i) the pump, (ii) the temperature control unit, and (iii) the channel of the portion of the article of furniture (e.g., the bed device). The pump may be configured to retrieve or receive the fluid from the container of the reservoir. The pump and/or the valve as provided herein may be configured to prevent flow of the fluid away from the pump and back into the container of the reservoir. The pump may be configured to direct flow of the fluid in the fluid loop, from the pump, to the temperature control unit, to the channel, and back to the pump. The temperature control unit may be configured to modulate a temperature of the fluid in the fluid loop. The system may comprise a second loop. The second fluid loop may comprise features that may or may not be identical to a first loop. The second fluid loop can comprise (i) the pump, (ii) the temperature control unit, and (iii) the channel of the portion of the article of furniture. The pump may be configured to retrieve or receive the fluid from the container of the reservoir. The pump (and/or a valve as provided herein) is configured to prevent flow of the fluid away from the pump and back into the container of the reservoir. The pump may be configured to direct flow of the fluid in the second fluid loop, from the pump, to the channel, to the temperature control unit, and back to the pump. The temperature control unit may be configured to modulate a temperature of the fluid in the second fluid loop. The system may further comprise one or more sensor(s) configured to detect a biological signal (e.g., a heart signal, a respiration signal, a motion, a temperature, and/or perspiration) of at least one user of the article of furniture. The one or more sensor(s) may be part of the article of furniture. The one or more sensor(s) and the portion of furniture may be located at different parts of the article of furniture. The system may regulate the temperature of the portion of furniture and/or the temperature of the portion of furniture based at least in part on the detected biological signal of the at least one user (e.g., one or two users) of the article of furniture. In some cases, one or more valves can control the flow (e.g., time, direction, flow rate, etc.) of the fluid between the container and any one of the pumps and. The valve(s) can be (i) disposed separately from the container, the reservoir, and the pumps, (ii) disposed as part of the container or the reservoir, or (iii) disposed as part of any one of the pumps. In some cases, the bed device may comprise a housing, which housing containing therewithin one or more members of the following: the reservoir, the container, the pump(s), the valve(s) between the container and the pump(s), the temperature control unit(s), and any fluid channels connecting there between. The furniture may not be contained within the housing.

Non-limiting examples of a pump as provided herein can include positive displacement pump, gear pump, screw pump, progressing cavity pump, roots-type pump, peristaltic pump, plunger pump, compressed-air-powered double-diaphragm pump, hydraulic pump, velocity pump, radial flow pump, axial flow pump, jet pump, gravity pump, steam pump, valveless pump, or any combination thereof.

Non-limiting examples of a valve as provided herein can include a check valve, clack valve, non-return valve, reflux valve, retention valve or one-way valve.

A pump and/or a valve (e.g., individually or a combination thereof) can operate as a flow controller (e.g., a gate) to control flow (e.g., time, direction, flow rate, etc.) of the fluid as provided herein. The gate can function as a one-way gate or a multi-way gate, such as two-way gate, three-way gate, four-way gate, etc.

In some cases, the temperature control unit can achieve temperature gelation via thermoelectric temperature regulation. Thermoelectric temperature regulation (e.g., heating and/or cooling) can be implemented using an electric-based system (e.g., by a thermoelectric engine). The thermoelectric engine can be configured to convert electrical energy into a heat flux (or a temperature difference), or convert the heat flux into electrical energy. The thermoelectric engine can be a solid-state device. In some examples, the temperature control unit can be a thermoelectric cooler, a thermoelectric heat pump, and/or a Peltier device.

Any of the systems and methods disclosed herein can be processed or performed (e.g., automatically) in real-time. The term “real time” or “real-time,” as used interchangeably herein, generally refers to an event (e.g., an operation, a process, a method, a technique, a computation, a calculation, an analysis, an optimization, etc.) that is performed using recently obtained (e.g., collected or received) data. Examples of the event may include, but are not limited to, receiving data from a source of the data (e.g., sensor, database, etc.), analyzing data, determining a conclusion or hypothesis based on the data, sending an instruction to a device (e.g., a temperature control unit), etc. In some cases, a real time event may be performed almost immediately or within a short enough time span, such as within at least 0.0001 ms, 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1ms, 0.05 ms, 0.01 ms, 0.005 ms, 0.001 ms, 0.0005 ms, 0.0001 ms, or less.

Other features of the article of furniture, such as a bed device, may be described in, for example, U.S. patent application Ser. No. 14/732,608 (“Methods and systems for gathering human biological signals and controlling a bed device”), U.S. patent application Ser. No. 14/732,624 (“Methods and systems for gathering and analyzing human biological signals”), U.S. patent application Ser. No. 15/178,124 (“Bed device system and methods”), Patent Cooperation Treaty Patent Application No. PCT/US2016/031054 (“An apparatus and methods for heating or cooling a bed based on human biological signals”), Patent Cooperation Treaty Patent Application No. PCT/US2016/030594 (“Vibrating alarm system and operating methods”), Patent Cooperation Treaty Patent Application No. PCT/US2019/014217 (“Sleep pod”), and Patent Cooperation Treaty Patent Application No. PCT/US2019/064056 (“Systems and methods for regulating a temperature of an article of furniture”), each of which is entirely incorporated herein by reference.

In some embodiments, a system comprising a bed device described herein can fluctuate in temperature. Temperature may fluctuate based on one or more biological signals of a user. In some embodiments, a temperature of a bed device and/or a portion of a bed device may fluctuate by at least about 0° C., at least about 1° C., at least about 2° C. at least about 3° C., at least about 4° C., at least about 5° C., at least about 6° C., at least about 7° C., at least about 8° C., at least about 9° C., at least about 10° C., at least about 11° C., at least about 12° C., at least about 13° C., at least about 14° C., at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., or greater than about 30° C. In some embodiments, a temperature of a bed device and/or a portion of a bed device may fluctuate by at most about at most about 30° C., at most about 29° C., at most about 28° C., at most about 27° C., at most about 26° C., at most about 25° C., at most about 24° C., at most about 23°C., at most about 22°C., at most about 21° C., at most about 20° C., at most about 19° C., at most about 18° C., at most about 17° C., at most about 16° C., at most about 15° C., at most about 14° C., at most about 13° C., at most about 12° C., at most about 11° C., at most about 10° C., at most about 9°C., at most about 8° C., at most about 7° C., at most about 6° C., at most about 5° C., at most about 4° C., at most about 3° C., at most about 2° C., at most about 1° C., or less than about 1°C.

In some embodiments, a temperature of a bed device and/or a portion of a bed device may fluctuate from about 1° C. to about 10° C. In some embodiments, a temperature of a bed device and/or a portion of a bed device may fluctuate from about 1° C. to about 2° C., about 1° C. to about 3° C., about 1°C. to about 4° C., about 1° C. to about 5° C., about 1° C. to about 6° C., about 1° C. to about 7° C., about 1°C. to about 8° C., about 1° C. to about 9° C., about 1° C. to about 10° C., about 2° C. to about 3° C., about 2 °C. to about 4° C., about 2° C. to about 5° C., about 2° C. to about 6° C., about 2° C. to about 7°C., about 2° C. to about 8° C., about 2° C. to about 9° C., about 2° C. to about 10° C., about 3° C. to about 4° C., about 3° C. to about 5° C., about 3° C. to about 6° C., about 3° C. to about 7° C., about 3° C. to about 8° C., about 3° C. to about 9° C., about 3° C. to about 10° C., about 4° C. to about 5° C., about 4° C. to about 6° C., about 4° C. to about 7° C., about 4° C. to about 8° C., about 4° C. to about 9° C., about 4° C. to about 10°C.., about 5° C. to about 6° C., about 5° C. to about 7° C., about 5° C. to about 8° C., about 5°C. to about 9° C., about 5° C. to about 10° C., about 6° C. to about 7° C., about 6° C. to about 8° C., about 6°C. to about 9°C., about 6°C. to about 10°C., about 7° C. to about 8° C., about 7° C. to about 9° C., about 7° C. to about 10° C., about 8° C. to about 9° C., about 8° C. to about 10° C., or about 9° C. to about 10° C. In some embodiments, a temperature of a bed device and/or a portion of a bed device may fluctuate from about 10° C. to about 40° C. In some embodiments, a temperature of a bed device and/or a portion of a bed device may fluctuate from about 10° C. to about 11° C., about 10° C. to about 12° C., about 10° C. to about 13° C., about 10° C. to about 14° C., about 10° C. to about 15° C., about 10° C. to about 20° C., about 10° C. to about 25°C., about 10° C. to about 30° C., about 10° C. to about 35° C., about 10° C. to about 40° C., about 11° C. to about 12° C., about 11° C. to about 13° C., about 11° C. to about 14°C., about 11° C. to about 15° C., about 11° C. to about 20° C., about 11° C. to about 25° C., about 11° C. to about 30° C., about 11° C. to about 35° C., about 11° C. to about 40° C., about 12° C. to about 13° C., about 12° C. to about 14° C., about 12° C. to about 15° C., about 12° C. to about 20° C., about 12° C. to about 25° C., about 12° C. to about 30° C., about 12° C. to about 35° C., about 12° C. to about 40° C., about 13° C. to about 14° C., about 13° C. to about 15° C., about 13° C. to about 20° C., about 13° C. to about 25° C., about 13° C. to about 30° C., about 13° C. to about 35° C., about 13° C. to about 40° C., about 14° C. to about 15° C., about 14° C. to about 20° C., about 14° C. to about 25° C., about 14° C. to about 30° C., about 14° C. to about 35° C., about 14° C. to about 40° C., about 15° C. to about 20° C., about 15° C. to about 25° C., about 15° C. to about 30° C., about 15° C. to about 35° C., about 15° C. to about 40° C., about 20° C. to about 25° C., about 20° C. to about 30° C., about 20° C. to about 35° C., about 20° C. to about 40° C., about 25° C. to about 30° C., about 25° C. to about 35° C., about 25° C. to about 40° C., about 30° C. to about 35° C., about 30° C. to about 40° C., or about 35° C. to about 40° C.

In some embodiments, the temperature control unit can be a part of (e.g., disposed inside or within) the article of furniture (e.g., part of the bed device, such as the mattress, mattress pad, pillow, or blanket). For example the temperature control unit can be disposed adjacent to or underneath the plurality of channels.

In some embodiments, the temperature control device being disposed adjacent to the plurality of channels may comprise the temperature control device being separated from the plurality of channels by at most about 10 centimeters (cm), at most about 9 cm, at most about 8 cm, at most about 7 cm, at most about 6 cm, at most about 5 cm, at most about 4 cm, at most about 3 cm, at most about 2 cm, at most about 1 cm, or less than about 1 cm.

In some embodiments, the system can comprise a sensor unit configured to detect one or more sensing data (e.g., one or more biological signals or absence thereof) associated with one or more users of the system (e.g., of the article of furniture comprising the system). The sensor unit can comprise a single sensor or a plurality of sensors that are the same or different. The sensor unit can be disposed on the top surface of the support member. The sensor unit can be disposed over the channel(s) that are placed into the groove(s) of the support member. Alternatively or in addition to, the sensor unit or an additional sensor unit can be disposed inside the support member, or underneath the bottom surface of the support member. In some cases, a first sensor unit comprising a first type of sensor for detecting a first type of sensing data (e.g., a capacitance sensor and/or a temperature sensor) can be disposed on the top surface of the support member, while a second sensor unit comprising a second type of sensor for detecting a second type of sensing data (e.g., a pressure sensor such as a piezo sensor) can be disposed under the bottom surface of the support member. For example, a sensor with high sensitivity (e.g., capacitive sensor) can be disposed on the top surface of the support member, while a sensor that is bulky (e.g., a pressure sensor such as a piezo sensor) can be disposed under the bottom surface of the support member, e.g., to reduce discomfort to the user during use of the system as provided herein.

In some embodiments, the sensor unit (e.g., disposed on top of the support member) can be a flexible sensor unit. The sensor unit can comprise a material (e.g., a polymeric material) that is flexible and/or can be made into a shape/geometry (e.g., a serpentine shape) to allow a degree of mechanical flexibility. Examples of such material can include an electrically conductive fabric (e.g., fabric coated with a conductive metal layer, such as silver, copper, or nickel), flexible printed circuit board assembly (PCBA), etc. The PCBA can comprise a polymeric substrate such as polyamide substrate.

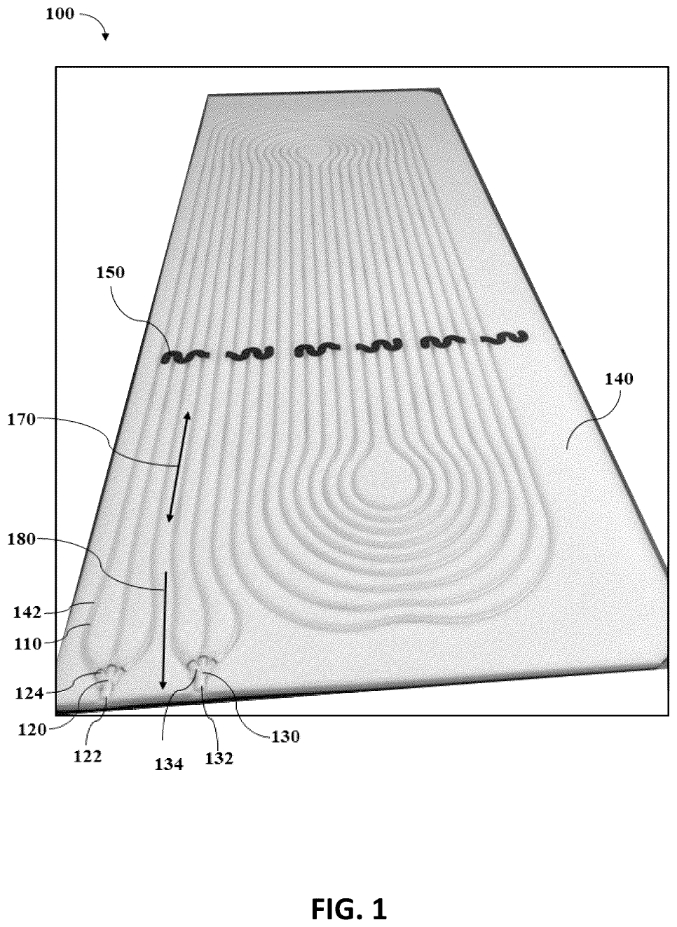

FIG. 1 schematically illustrates an isometric view of an example system 100 of the present disclosure. The system 100 can be configured to be a part of an article of furniture such as a bed. The system 100 can comprise a plurality of channels (e.g., TPE tubes) 110 for permitting flow of fluid (e.g., liquid) that is temperature regulated. The system can comprise a first junction 120 that is fluidically coupled to a source of the fluid via the inlet port 122. The first junction 120 is also fluidically coupled to the channels 110 via outlet ports 124, to direct flow of the fluid from the source and towards the channels 110. The other ends of the channels are fluidically coupled to a second junction 130 via the inlet ports 134. The second junction 130 is then fluidically coupled to (i) the source and/or (ii) the first junction 120, to promote additional or continuous flow of the fluid throughout the channels. The system 100 can comprise a support member (e.g., a foam) 140 comprising a plurality of grooves 142. The plurality of channels 110 can be laid into the plurality of grooves 142 of the support member 140, such that the plurality of channels 110 does not protrude out of a plane of the top surface of the support member 140. The system can further comprise one or more sensor units (e.g., one or more sensor layers, such as sensor strips) 150 comprising one or more sensors. The sensor unit(s) 150 can be disposed over a top surface of the support member 140 and over the grooves 142 of the support member 140.

For example, the plurality of channels 110 can be laid into the plurality of grooves 142 of the support member 140 such that the plurality of channels protrude out of the plane of the top surface of the support member by at most about 50 cm, at most about 40 cm, at most about 30 cm, at most about 20 cm, at most about 10 cm, at most about 9 cm, at most about 8 cm, at most about 7 cm, at most about 6 cm, at most about 5 cm, at most about 4 cm, at most about 3 cm, at most about 2 cm, at most about 1 cm, or less than about 1 cm. The plurality of channels 110 can be laid into the plurality of grooves 142 of the support member 140 such that the plurality of channels protrude out of the plane of the top surface of the support member by an amount from about 1 cm to about 20 cm. The plurality of channels 110 can be laid into the plurality of grooves 142 of the support member 140 such that the plurality of channels protrude out of the plane of the top surface of the support member by an amount from about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 9 cm, about 1 cm to about 10 cm, about 1 cm to about 15 cm, about 1 cm to about 20 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 2 cm to about 15 cm, about 2 cm to about 20 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 3 cm to about 15 cm, about 3 cm to about 20 cm, about 4 cm to about 5 cm, about 4 cm to about 6 cm, about 4 cm to about 7 cm, about 4 cm to about 8 cm, about 4 cm to about 9 cm, about 4 cm to about 10 cm, about 4 cm to about 15 cm, about 4 cm to about 20 cm, about 5 cm to about 6 cm, about 5 cm to about 7 cm, about 5 cm to about 8 cm, about 5 cm to about 9 cm, about 5 cm to about 10 cm, about 5 cm to about 15 cm, about 5 cm to about 20 cm, about 6 cm to about 7 cm, about 6 cm to about 8 cm, about 6 cm to about 9 cm, about 6 cm to about 10 cm, about 6 cm to about 15 cm, about 6 cm to about 20 cm, about 7 cm to about 8 cm, about 7 cm to about 9 cm, about 7 cm to about 10 cm, about 7 cm to about 15 cm, about 7 cm to about 20 cm, about 8 cm to about 9 cm, about 8 cm to about 10 cm, about 8 cm to about 15 cm, about 8 cm to about 20 cm, about 9 cm to about 10 cm, about 9 cm to about 15 cm, about 9 cm to about 20 cm, about 10 cm to about 15 cm, about 10 cm to about 20 cm, or about 15 cm to about 20 cm.

FIG. 7 schematically illustrates an example of the sensor 150. The sensor can be a flexible sensor having a serpentine shape. The sensor can comprise a top conductive layer 710 (e.g., top copper layer) and a bottom conductive layer 720 (e.g., bottom copper layer).

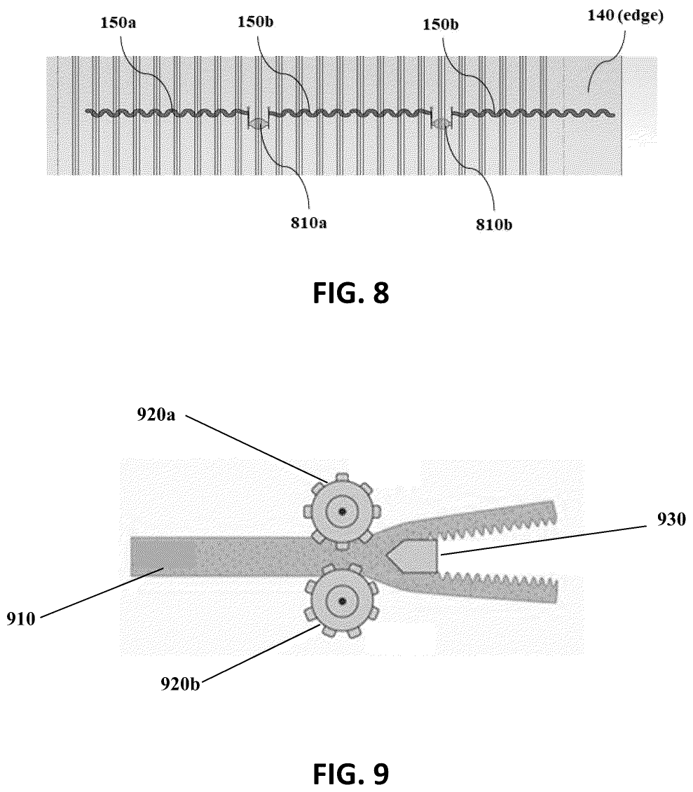

FIG. 8 schematically illustrates a top view of the example system 100 as shown in FIG. 1. As shown in FIG. 8, three flexible serpentine sensors 150a, 150b, and 150c can be disposed on top of the support member 140, and the sensors 150a, 150b, and 150c can be electrically and/or digitally connected to one another via the junctions 810a and 810b. The junctions 810a and 810b can each comprise a printed circuit board (PCB) that are in digital communication (e.g., via wires or wireless communications such as Wi-Fi or Bluetooth) with an analog-to-digital converter (ADC) and/or a processor capable of handling (e.g., processing, analyzing, etc.) sensing data from the sensors 150a, 150b, and 150c and/or from one or more additional sensors in the PCB (e.g., temperature sensor, piezo sensor, etc.). For example, the PCB can comprise a temperature sensor to detect sensing data indicative of (i) a temperature of the system adjacent to the temperature sensor or (ii) a temperature of a user adjacent to or on top of the temperature sensor. One or both of the junctions 810a and 810b can be disposed under, within, or on top of the support member 140.

FIG. 2 schematically illustrates a cross-sectional view of the support member (e.g., a foam) of the example system 100 as shown in FIG. 1. As shown in FIG. 2, the support member 140 comprises a plurality of grooves 142 to each hold or substantially house a channel (e.g., a tube). FIG. 3 schematically illustrates channels 110 is nested inside each of the groove 142 (and thus nested inside the support member 140) without protruding outside the top surface of the support member 140.

In some embodiments, a first portion (e.g., an inner region) of the support member having the channel embedded within the groove (e.g., the inner region as indicated by the arrow 170 in FIG. 1) can have a thickness that is relative to the thickness of the channel. For example, the channel has a thickness that is substantially constant along its length (e.g., along the length of flow of the fluid inside the channel), and accordingly, the first portion of the support member having the channel embedded within the groove can have a support member thickness that is substantially the same along the length of the groove.

In some embodiments, a second portion (e.g., an outer region or a region near the edge) of the support member having (or being disposed on top of) additional component(s) of the fluidic system other than the channels can exhibit a property (e.g., thickness and/or stiffness) that is different than that of the first portion (e.g., the inner region) of the support member. For example, the second portion of the support member can be the outer region as indicated by the arrow 180 in FIG. 1. Also, as illustrated in FIG. 1, non-limiting examples of the additional component(s) of the fluidic system can include the first junction 120, the inlet port 122, the outlet port 124, or any other channels that are directly coupled to or in fluid communication with the source of the fluid, which source is disposed outside of the support member, etc. In some cases, the additional component(s) of the fluidic system can be thicker and/or stiffer than the channel, and thus the second portion of the support member configured to contain, cover, or be adjacent to the additional component(s) of the fluidic system can be thicker and/or stiffer-as compared to the first portion of the support member configured to contain just the channel-to substantially “hide” the thickness and/or stiffness difference from the user when the user is on top of the system. In some cases, the support member can exhibit a gradual difference (e.g., increase) in its thickness and/or stiffness along the direction from the inner portion and towards the outer portion (e.g., as indicated by the direction of the arrow 180 as shown in FIG. 1).

In some embodiments, the thickness and/or stiffness of the support member can be substantially uniform across its dimension (e.g., length, width).

In some embodiments, an average thickness and/or stiffness of the second (or outer/edge) portion of the support member can be greater than the average thickness and/or stiffness of the first (or inner) portion of the support member by at least or at most about 1%, at least or at most about 2%, at least or at most about 5%, at least or at most about 10%, at least or at most about 15%, at least or at most about 20%, at least or at most about 25%, at least or at most about 30%, at least or at most about 40%, at least or at most about 50%, at least or at most about 60%, at least or at most about 70%, at least or at most about 80%, at least or at most about 90%, at least or at most about 100%, at least or at most about 120%, at least or at most about 150%, at least or at most about 200%, at least or at most about 250%, at least or at most about 300%, at least or at most about 400%, or at least or at most about 500%.

In some embodiments, the increased stiffness of the second (or outer/edge) portion can be achieved by having a higher density of the material that makes up the support member (e.g., foam). The average density of the second (or outer/edge) portion of the support member can be greater than the average density of the first (or inner) portion of the support member by at least or at most about 1%, at least or at most about 2%, at least or at most about 5%, at least or at most about 10%, at least or at most about 15%, at least or at most about 20%, at least or at most about 25%, at least or at most about 30%, at least or at most about 40%, at least or at most about 50%, at least or at most about 60%, at least or at most about 70%, at least or at most about 80%, at least or at most about 90%, at least or at most about 100%, at least or at most about 120%, at least or at most about 150%, at least or at most about 200%, at least or at most about 250%, at least or at most about 300%, at least or at most about 400%, or at least or at most about 500%.

In some embodiments, the change in the property (e.g., thickness, stiffness, density, etc.) of the support member can be abrupt or gradual. For example, the gradual change of the property (e.g., as measured per length along the direction of the arrow 180 as shown in FIG. 1) can be at least or at most about 1% per unit length (e.g., millimeter or centimeter), at least or at most about 2% per unit length, at least or at most about 3% per unit length, at least or at most about 4% per unit length, at least or at most about 5% per unit length, at least or at most about 6% per unit length, at least or at most about 7% per unit length, at least or at most about 8% per unit length, at least or at most about 9% per unit length, at least or at most about 10% per unit length, at least or at most about 11% per unit length, at least or at most about 12% per unit length, at least or at most about 13% per unit length, at least or at most about 14% per unit length, at least or at most about 15% per unit length, at least or at most about 20% per unit length, at least or at most about 25% per unit length, at least or at most about 30% per unit length, at least or at most about 40% per unit length, at least or at most about 50% per unit length, at least or at most about 60% per unit length, at least or at most about 70% per unit length, at least or at most about 80% per unit length, at least or at most about 90% per unit length, or at least or at most about 100% per unit length.

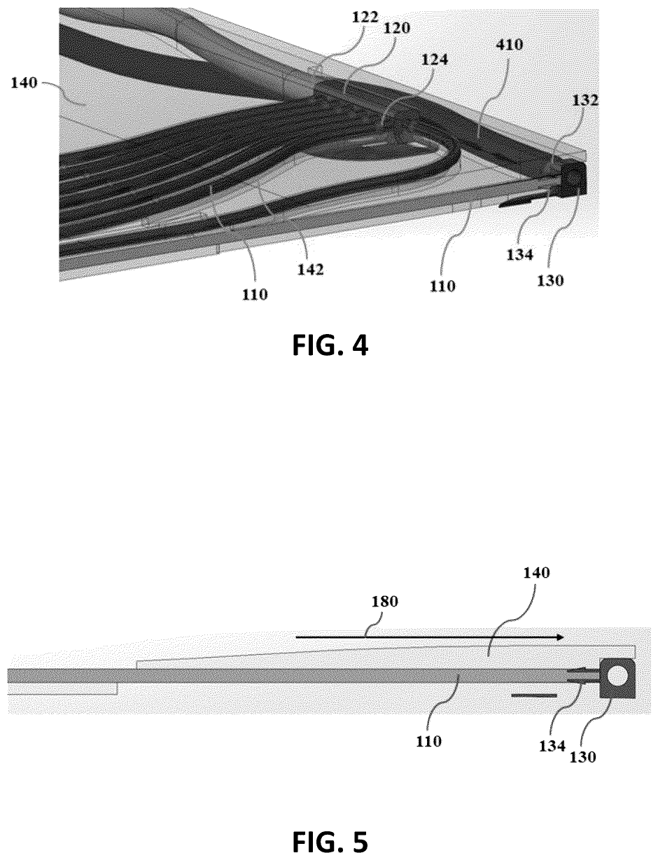

FIG. 4 illustrates a semi-transparent isometric view of the example system 100 comprising the components as illustrated in FIG. 1 in addition to other components such as FIG. 5 schematically illustrates a cross-sectional side view of the example system 100, to demonstrate the increase in thickness of the support member 140 along the direction from an inner portion and towards an edge portion of the support member 140, which direction is indicated by the arrow 180.

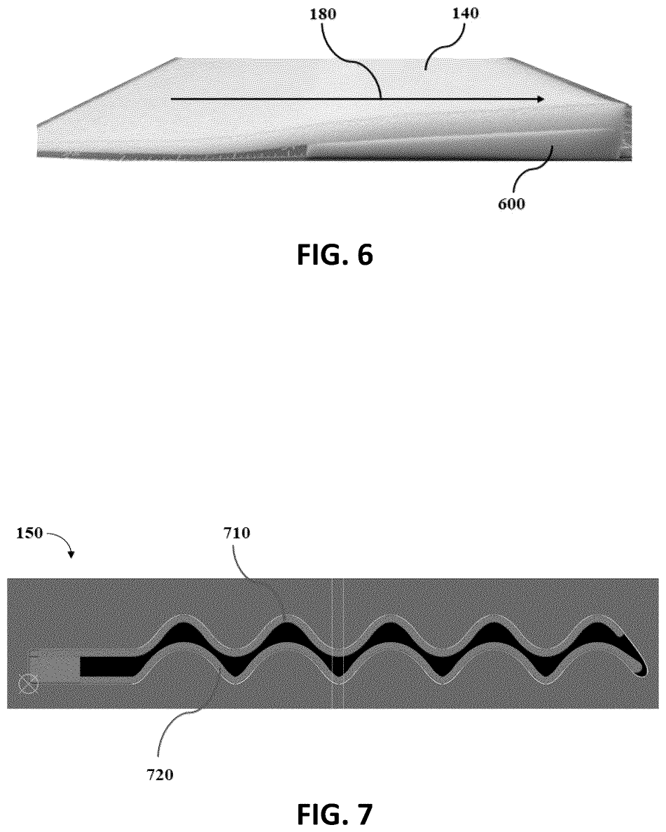

In some embodiments, the stiffness of the second portion (e.g., the outer or edge region) of the support member can be changed at least by having an additional layer of the support member adjacent to (e.g., over or underneath) the second portion of the support member. The support member can comprise a first layer that is continuous between the first portion (e.g., the inner portion) and the second portion, and the support member can comprise a second layer adjacent to the first layer. The first layer and the second layer can have the same or different materials, dimensions (e.g., thickness, length, etc.), and/or properties (e.g., stiffness, density, etc.). For example, as illustrated in FIG. 6, the support member can comprise a first layer 140 that is disposed on top of (e.g., overlaying) a second layer 600, which second layer 600 is serving as a stiffener layer to increase the overall stiffness or rigidness of the support member towards the edge where other thicker or bulkier components of the fluidic system would be located. The second layer of the support member can exhibit a change of a dimension (e.g., thickness), and/or property (e.g., stiffness, density, etc.) along the direction as indicated by the arrow 180 as shown in FIG. 6, as described herein in the context of the support member.

In some embodiments, the average thickness of the first layer of the support member (e.g., an average thickness of the edge region of the first layer) can be less than, equal to, or greater than the average thickness of the second layer of the support member. The first layer can be disposed on top of the second layer, and the average thickness of the first layer can be different from (e.g., greater than or less than) the average thickness of the second layer by at least or at most about 1%, at least or at most about 2%, at least or at most about 3%, at least or at most about 4%, at least or at most about 5%, at least or at most about 6%, at least or at most about 7%, at least or at most about 8%, at least or at most about 9%, at least or at most about 10%, at least or at most about 15%, at least or at most about 20%, at least or at most about 25%, at least or at most about 30%, at least or at most about 35%, at least or at most about 40%, at least or at most about 45%, at least or at most about 50%, at least or at most about 60%, at least or at most about 70%, at least or at most about 80%, at least or at most about 90%, at least or at most about 95%, or at least or at most about 100%. For example, the average thickness of the edge portion of the first layer can be greater than the average thickness of the second layer, e.g., by between about 20% and about 50%.

In some embodiments, the average property (e.g., stiffness, density, etc.) of the first layer of the support member (e.g., an average thickness of the edge region of the first layer) can be less than, equal to, or greater than the average property of the second layer of the support member. The first layer can be disposed on top of the second layer, and the average property of the first layer can be different from (e.g., greater than or less than) the average property of the second layer by at least or at most about 1%, at least or at most about 2%, at least or at most about 3%, at least or at most about 4%, at least or at most about 5%, at least or at most about 6%, at least or at most about 7%, at least or at most about 8%, at least or at most about 9%, at least or at most about 10%, at least or at most about 15%, at least or at most about 20%, at least or at most about 25%, at least or at most about 30%, at least or at most about 35%, at least or at most about 40%, at least or at most about 45%, at least or at most about 50%, at least or at most about 60%, at least or at most about 70%, at least or at most about 80%, at least or at most about 90%, at least or at most about 95%, or at least or at most about 100%. For example, the average density of the second layer can be greater than the average density of the edge portion of the first layer, e.g., by at least about 75%.

In some embodiments, the thickness of the support member (e.g., the first layer or the second layer as provided herein) can be at least or at most about 1 millimeter, at least or at most about 2 millimeters, at least or at most about 3 millimeters, at least or at most about 4 millimeters, at least or at most about 5 millimeters, at least or at most about 6 millimeters, at least or at most about 7 millimeters, at least or at most about 8 millimeters, at least or at most about 9 millimeters, at least or at most about 10 millimeters, at least or at most about 11 millimeters, at least or at most about 12 millimeters, at least or at most about 13 millimeters, at least or at most about 14 millimeters, at least or at most about 15 millimeters, at least or at most about 16 millimeters, at least or at most about 17 millimeters, at least or at most about 18 millimeters, at least or at most about 19 millimeters, at least or at most about 20 millimeters, at least or at most about 21 millimeters, at least or at most about 21 millimeters, at least or at most about 23 millimeters, at least or at most about 24 millimeters, at least or at most about 25 millimeters, at least or at most about 26 millimeters, at least or at most about 27 millimeters, at least or at most about 28 millimeters, at least or at most about 29 millimeters, at least or at most about 30 millimeters, at least or at most about 35 millimeters, at least or at most about 40 millimeters, at least or at most about 45 millimeters, or at least or at most about 50 millimeters.

In some embodiments, the material density of the support member (e.g., the first layer or the second layer as provided herein) can be at least or at most about 5 kilograms per cubic meters, at least or at most about 10 kilograms per cubic meters, at least or at most about 15 kilograms per cubic meters, at least or at most about 20 kilograms per cubic meters, at least or at most about 25 kilograms per cubic meters, at least or at most about 30 kilograms per cubic meters, at least or at most about 35 kilograms per cubic meters, at least or at most about 40 kilograms per cubic meters, at least or at most about 45 kilograms per cubic meters, at least or at most about 50 kilograms per cubic meters, at least or at most about 60 kilograms per cubic meters, at least or at most about 70 kilograms per cubic meters, at least or at most about 80 kilograms per cubic meters, at least or at most about 90 kilograms per cubic meters, or at least or at most about 100 kilograms per cubic meters. For example, the material density of the support member (e.g., a polyurethane foam) can range between about 10 kilograms per cubic meters and about 40 kilograms per cubic meters, between about 20 kilograms per cubic meters and about 30 kilograms per cubic meters, or between about 23 kilograms per cubic meters and about 28 kilograms per cubic meters.

In some embodiments, the stiffness of the support member (e.g., the first layer or the second layer as provided herein), as measured by indentation load deflection (ILD) method or indentation force deflection (IFD) method. In some embodiments, the indentation load deflection (ILD) can be a measure of hardness defined in an ISO standard (e.g., ISO 2439 standard). In some embodiments, the ILD can be a measure of hardness defined in an ASTM standard (e.g., ASTM D3574 B1). In some cases, the standard defines ILD as the force that is required to compress the foam to a specified percentage of its original thickness using a circular plate of fifth sequence inches. In some embodiments, a 20%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 25%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 30%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 35%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 40%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 45%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 50%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 55%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 60%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 65%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 70%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. In some embodiments, a 75%-compression ILD of the support member (e.g., the first layer or the second layer as provided herein) can be at least or at most about 5 pound-force, at least or at most about 10 pound-force, at least or at most about 15 pound-force, at least or at most about 20 pound-force, at least or at most about 25 pound-force, at least or at most about 30 pound-force, at least or at most about 35 pound-force, at least or at most about 40 pound-force, at least or at most about 45 pound-force, or at least or at most about 50 pound-force. For example, the stiffness of the support member (e.g., a polyurethane foam) can range between about 10 pound-force and about 35 pound-force, between about 15 pound-force and about 30 pound-force, or between about 20 pound-force and about 25 pound-force.

For example, as illustrated in FIG. 6, the outer portion of the support member can comprise (i) a first top layer 140 having a thickness ranging between about 10 millimeters and 14 millimeters and a material density of about 28 kilograms per cubic meters and (ii) a second bottom “stiffener” layer having a thickness of about 6 millimeters and a material density of about 50 kilograms per cubic meters.

In some embodiments, the support member may not and need not exhibit any change in its dimension (e.g., thickness) and/or property (e.g., stiffness, density, etc.) along the direction from an inner portion and towards the outer portion (e.g., edge) of the support member. In some embodiments, the first layer and/or the second layer of the support member may not and need not exhibit any change in its dimension (e.g., thickness) and/or property (e.g., stiffness, density, etc.) along the direction from an inner portion and towards the outer portion (e.g., edge) of the first layer and/or the second layer of the support member.

In some embodiments, patterns (e.g., the one or more grooves) of the support member (e.g., foam) can be generated by removing (e.g., cutting away) one or more portions of a non-patterned support member to generate the support member having such textural patterns. The patterns can be two-dimensional or three-dimensional, such as the one or more grooves as provided herein. For example, the non-patterned support member can be a foam layer having a substantially flat top surface.

In some embodiments, the patterned support member can be generated via a rotary convolution cut process. The patterned support member can comprise a foam material (910). The non-patterned support member can be directed through two circular rollers (920a and 920b) having complementary patterns (e.g., corresponding to the desired pattern of the support member) while the circular rollers (920a and 920b) are rolling along its rolling axis (e.g., perpendicular to the radial direction of the circular surface). Subsequently or substantially at the same time, the portion of the non-patterned support member that is partly compressed and deformed by the circular rollers are pushed through a divider (930). In some embodiments, the divider may be a flat blade). The divider can cut the non-patterned support member into two patterned halves. The at least one of the two halves may be used as a patterned support member as provided herein. In such case, the divider remains substantially stationary while the non-patterned support member is moving across the divider. FIG. 9 schematically illustrates the rotary convolution cut process as described herein.

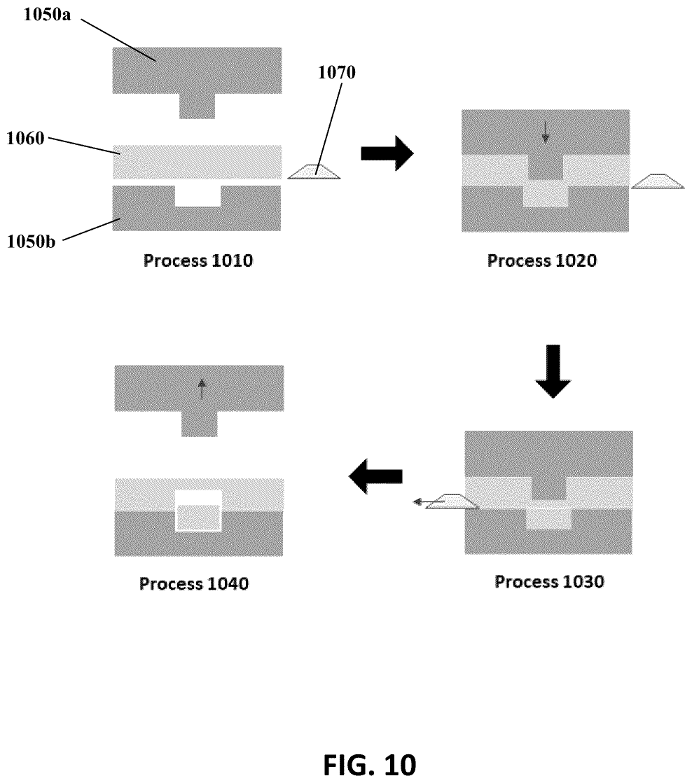

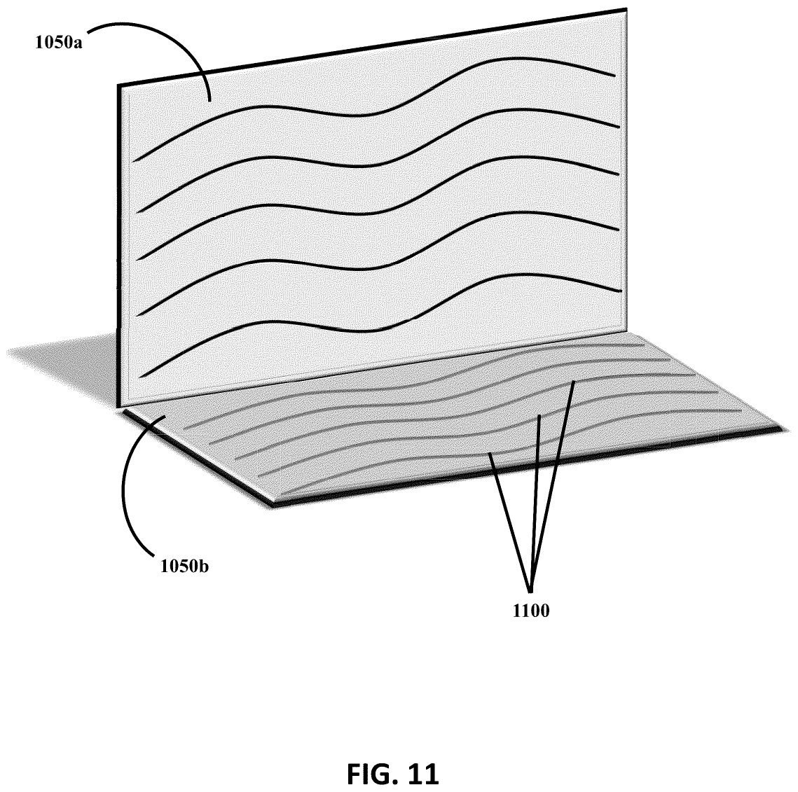

In some embodiments, the desired pattern (e.g., pattern of the one or more grooves) on the support member may not be a uniform and/or repeating pattern, and the rotary convolution cut process utilizing patterned rollers may not be capable of creating such non-uniform and/or repeating pattern on the support member. Accordingly, the patterned support member can be generated via a non-rotary convolution cut process. To generate the patterns (e.g., cut one or more grooves) in the support member, two substantially flat molds can be generated, comprising a first mold having the desired pattern (e.g., shape) and a second mold having a negative image of the desired pattern. A non-patterned support member can be sandwiched between the first mold and the second mold, then compressed between the two molds, to deform the non-patterned support member in accordingly with the patterns of the two molds. Subsequently, the compressed non-patterned support member and a blade can move relatively towards one another, to permit the blade to cut across the compressed non-patterned support member, to generate a patterned support member. During the relative movement between the compressed non-patterned support member and the blade, the moving pieces may be the compressed non-patterned support member, the blade, or both. In such process, the molds are substantially flat, e.g., not circular rollers that can rotate around its rolling axis. FIG. 10 schematically illustrates the non-rotary convolution cut process as described herein. FIG. 11 schematically illustrates an example of two molds for the non-rotary convolution cut process as shown in FIG. 10.

The non-rotary convolution cut procedure can comprise a first process (1010). The first process (1010) can comprise a top mold (1050a) and/or a bottom mold (1050b). The foam (1060) can be placed onto the bottom mold (1050b). The bottom mold (1050b) can comprise one or more grooves (e.g., 1, 2, 3, 4, 5, or more grooves). A blade (1070) may be external to the top mold and bottom mold. In a second process (1020), the top mold (1050a) may come down (e.g., lower) onto the foam (1060). The top mold (1050a) can compress the foam (1060) into a groove. In the third process (1030), the blade (1070) may move through the foam (1060) and cut the foam. The blade (1070) may travel along an axis of the bottom mold (1050b) to cut the foam (1060). In a fourth process (1040), the tool comprising the top mold (1050a) and/or bottom mold (1050b) may open. The tool may open once the blade (1070) has passed through the foam (1060). Once the tool opens, the foam (1060) may then expand to provide an expanded foam. The groove cut into the foam may be revealed. In some embodiments, following the first process (1010), second process (1020), third process (1030), fourth process (1040), or any combination thereof, the foam (1060) may comprise at least about 1 groove, at least about 2 grooves, at least about 3 grooves, at least about 4 grooves, at least about 5 grooves, at least about 10 grooves, or greater than about 10 grooves. FIG. 11 shows the top mold (1050a) and bottom mold (1050b) separated. The grooves (1100) are shown in the foam of the support member. These may be desired patterns.

In some embodiments, during the non-rotary convolution cut process, the compression force exerted on the non-patterned support member via one or both of the top and bottom molds can be at least or at most about 100 pound-force, at least or at most about 200 pound-force, at least or at most about 300 pound-force, at least or at most about 400 pound-force, at least or at most about 500 pound-force, at least or at most about 600 pound-force, at least or at most about 700 pound-force, at least or at most about 800 pound-force, at least or at most about 900 pound-force, at least or at most about 1,000 pound-force, at least or at most about 1,200 pound-force, at least or at most about 1,500 pound-force, at least or at most about 2,000 pound-force, at least or at most about 2,500 pound-force, at least or at most about 3,000 pound-force, at least or at most about 4,000 pound-force, at least or at most about 5,000 pound-force, or at least or at most about 10,000 pound-force.

In some embodiments, during the non-rotary convolution cut process, the non-patterned support member may be lubricated by a lubricant. The lubricant can be applied to the non-patterned support member (e.g., on the top surface and/or the bottom surface) prior to, during, or subsequent to being compressed between the top mold and the bottom mold. The lubricant can be applied to the non-patterned support member prior to, during, or subsequent to being cut by the blade. Non-limiting examples of the lubricant can include mineral oil (e.g., hydrocarbon-based oil such as fossil fuel-based oil), synthetic oil (e.g., hydrogenated polyolefins, polyalkaline glycols, esters, silicone, fluorocarbons), and vegetable oil (e.g., seed oil).

In some embodiments, the lubricant can comprise a single type of oil or a plurality of different oils (e.g., a mineral oil and a synthetic oil, such as silicone oil). In some embodiments, the lubricant can be a mixture comprising an oil (e.g., a synthetic oil, such as silicone oil) and a non-oil solvent such as, for example, an organic solvent. Non-limiting examples of the organic solvent can include ethanol, methanol, ether (e.g., diethyl ether), chloroform, alcohol, and acetone. For example, a hybrid lubricant can comprise a synthetic oil (e.g., silicone oil) and an organic solvent (e.g., ether). In some cases, presence of the organic solvent can promote evaporation of at least a portion of the lubricant, thereby reducing or preventing disruption of any subsequent manufacturing process of the patterned support member by excess amount of residual lubricant. For the hybrid lubricant comprising an oil and an organic solvent, a ratio (e.g., volume ratio, weight ratio, etc.) between the oil and the organic solvent can be at least or at most about 100:1, at least or at most about 95:1, at least or at most about 90:1, at least or at most about 85:1, at least or at most about 80:1, at least or at most about 75:1, at least or at most about 70:1, at least or at most about 65:1, at least or at most about 60:1, at least or at most about 55:1, at least or at most about 50:1, at least or at most about 45:1, at least or at most about 40:1, at least or at most about 35:1, at least or at most about 30:1, at least or at most about 25:1, at least or at most about 20:1, at least or at most about 15:1, at least or at most about 10:1, at least or at most about 9:1, at least or at most about 8:1, at least or at most about 7:1, at least or at most about 6:1, at least or at most about 5:1, at least or at most about 4:1, at least or at most about 3:1, at least or at most about 2:1, at least or at most about 1:1, at least or at most about 1:2, at least or at most about 1:2, at least or at most about 1:3, at least or at most about 1:4, at least or at most about 1:5, at least or at most about 1:6, at least or at most about 1:7, at least or at most about 1:8, at least or at most about 1:9, at least or at most about 1:10, at least or at most about 1:15, at least or at most about 1:20, at least or at most about 1:25, at least or at most about 1:30, at least or at most about 1:35, at least or at most about 1:40, at least or at most about 1:45, at least or at most about 1:50, at least or at most about 1:55, at least or at most about 1:60, at least or at most about 1:65, at least or at most about 1:70, at least or at most about 1:75, at least or at most about 1:80, at least or at most about 1:85, at least or at most about 1:90, at least or at most about 1:95, or at least or at most about 1:100.

In some embodiments, the patterned support member can be generated by casting liquid starting material (e.g., liquid foam) into a mold, and subsequently solidifying and/or hardening the liquid starting material. In some embodiments, a size of the patterned support member (e.g., the size of a full bed, double bed, queen bed, king bed, California king bed, etc.) may be too big to utilize the liquid starting material/mold process, and thus the non-rotary convolution cut process may be utilized.

In some embodiments, the patterned support membrane can be generated by laying the fluidic system (e.g., at least the channels such as tubes) in to a mold for the support member, and subsequently pouring the liquid foam material onto the fluidic system. In some cases, the liquid foam material can solidify and/or harden into the mold.

In some embodiments, subsequent to (i) disposing the fluidic system (e.g., at least the channels such as tubes) into the grooves of the support member and optionally (ii) disposing the one or more sensor units on the top surface of the support member and over at least a portion of the fluidic system, a protective layer (e.g., a fabric layer) can be disposed over at least a portion of the top surface of the support member to cover the support member, the channels, and the sensor units. In some cases, an adhesive material (e.g., glue) can be applied to at least a portion of the support member, such that the protective layer can be attached to the at least the portion of the support member. In some cases, the adhesive material may not be applied to the channels and/or the sensor units. Alternatively, the adhesive material may be applied to at least a portion of an outer surface of the channels and/or at least a portion of a top surface of the sensor units.

In some embodiments, the protective layer and/or the support member can be stretched (e.g., along a single direction or multiple directions such as at least along two substantially orthogonal directions) when placing the protective layer over the support member. The member being stretched out can be just the protective layer, just the support member, or both. In some cases, an aspect of the present disclosure provides a system for coupling the protective layer to the support member as described herein. The system can comprise one or more coupling mechanisms (e.g., one or more clips) to hold the protective layer, to stretch the protective layer. For example, the system can comprise a plurality of coupling mechanisms (e.g., a coupling mechanism on each side of the four sides of the protective layer) to strength out the protective layer along a plurality of direction (e.g., along the four sides of the protective layer). Subsequently, a relative movement between the protective layer (e.g., while being stretched out by the coupling mechanism(s)) and the support member can promote adhesion between the protective layer and the support member. Alternatively or in addition to, similar coupling mechanisms can be utilized to stretch out the support member if needed.

In some embodiments, the protective layer and/or the support member may not and need not be stretched along one or more directions (e.g., may not be stretched along any direction) when placing the protective layer over the support member. In some cases, not stretching the protective layer during such process can reduce or avoid deformation (e.g., curling) of the support member.

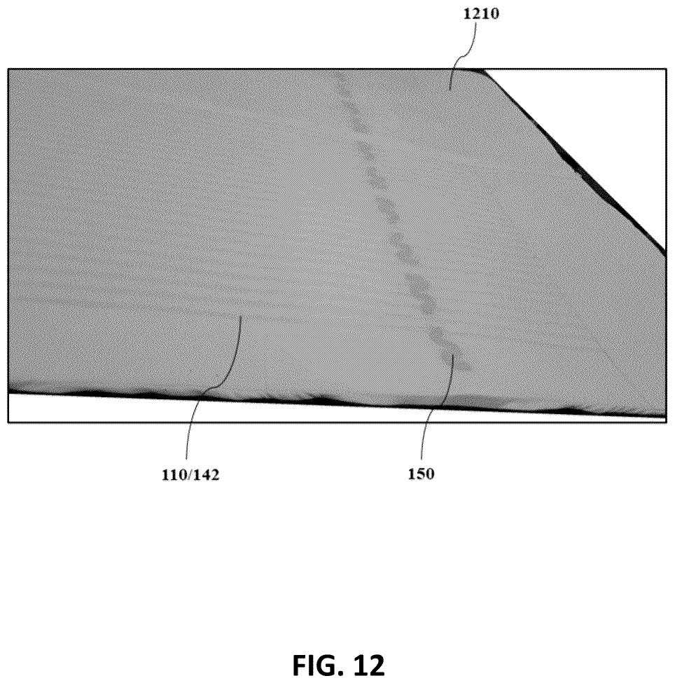

FIG. 12 shows an image of the protective layer (e.g., fabric) glued onto the top surface of the support member, thereby covering over the sensor unit(s) 150 and the channel(s) 110 that are disposed within the groove(s) 142 of the support member. In some cases, the protective layer can prevent the channels from coming out of the grooves.

The present disclosure provides systems and methods for allowing a user of an article of furniture to control one or more of the following while using the article of furniture (e.g., while being on or on top of the article of furniture): (i) a condition of the article of furniture, (ii) a condition of an environment comprising the article of furniture, (iii) a user device of the user, and/or (iv) other devices in the environment or associated with the user.

In some embodiments, the system can comprise the article of furniture comprising a sensor unit in a sensor zone of the article of furniture. The sensor unit can be configured to, upon activation (e.g., via direct or indirect contact) by the user, control (i) the condition of the article of furniture, (ii) the condition of the environment, (iii) the user device, and/or (iv) the other devices. In some embodiments, the user of the article of furniture can contact the sensor zone (e.g., tap one or more regions of the sensor zone, swipe over at least a portion of the sensor zone) to control (i) the condition of the article of furniture, (ii) the condition of the environment, (iii) the user device, and/or (iv) other devices, e.g., without having to utilize other devices (e.g., a remote control device of the article of furniture, a user device such as a mobile phone operatively coupled to the article of furniture, etc.). The sensor zone can be an area that is not in contact (e.g., physical contact) with the user during the user's normal use of the article of furniture. In some cases, when the use of the article of furniture requires the user to sit lay on top of the article of furniture, the sensor zone of the article of furniture may not be the portion of the article of furniture that is configured to be in contact with the user's feet, legs, torso, and/or head.

In some embodiments, the sensor unit can comprise one or more sensors, e.g., a single sensor, a plurality of sensors of the same type, a plurality of sensors of different types, etc. The sensor(s) can comprise a contact sensor or a touch sensor, which terms may be used interchangeably herein. A contact sensor can be capable of detecting contact and/or pressure (e.g., such as that of a hand of a user of the article of furniture). Non-limiting examples of a contact sensor can include a sensor capable of detecting changes in magnetism, conductivity, temperature, pressure, capacitance, and/or resistance. Alternatively or in addition to, the sensor(s) can comprise an accelerometer. The accelerometer can be configured to measure acceleration, vibration, and/or movement (e.g., such as that of a hand of a user of the article of furniture). Non-limiting examples of the accelerometer can include an accelerometer, gyroscope, magnetometer, etc. In some cases, the sensor unit can comprise a plurality of sensors of different types, such as a capacitive sensor and an accelerometer.

In some embodiments, the sensor unit can be configured (or programmed) to detect and identify different interactions of the user, such as different durations of touch by the user, different pressure of the touch by the user, and/or different swiping motions of the user (e.g., swipe up, down, left, right, at least partial rotation, back-and-forth, a combination there of, etc.). The duration can be at least or at most about 0.1 seconds, at least or at most about 0.5 seconds, at least or at most about 1 second, at least or at most about 1.5 seconds, at least or at most about 2 seconds, at least or at most about 3 seconds, at least or at most about 4 seconds, at least or at most about 5 seconds, at least or at most about 6 seconds, at least or at most about 7 seconds, at least or at most about 8 seconds, at least or at most about 9 seconds, or at least or at most about 10 seconds. The sensor unit can be programmed such that each interaction can be translated to a specific implementation of controlling (i) the condition of the article of furniture, (ii) the condition of the environment, (iii) the user device, and/or (iv) other devices.

In some embodiments, the sensor unit can comprise a housing configured to contain at least a portion of the sensor(s) as provided herein.

In some embodiments, the article of furniture can comprise (i) a first surface configured to be in contact (e.g., physical contact) with the user during the user's normal or ordinary use of the article of furniture (e.g., sleeping on top of a bed or a pillow, sitting on a chair, etc.) and (ii) a second surface that is not configured to be in contact with the user during the users normal/ordinary use of the article of furniture. The sensor zone can be a part of the second surface. The first surface and the second surface can be different surfaces. The first surface and the second surface may or may not be parallel to one another. The first surface and the second surface can be offset from one another by at least or at most about 1 degree, at least or at most about 5 degrees, at least or at most about 10 degrees, at least or at most about 15 degrees, at least or at most about 20 degrees, at least or at most about 30 degrees, at least or at most about 40 degrees, at least or at most about 50 degrees, at least or at most about 60 degrees, at least or at most about 70 degrees, at least or at most about 80 degrees, or at least or at most about 90 degrees. In some cases, the first surface and the second surface can be substantially perpendicular to one another. For example, the first surface can be a top surface of the article of furniture, and the second surface can be a side surface of the article of furniture.

In some embodiments, the sensor zone can be limited to a portion of the second surface of the article of furniture that is within arms-length of the user while the user is using the article of furniture (e.g., when the user is on top of the first surface of the article of furniture). For example, the sensor zone can be near or adjacent to a portion of the first surface that corresponds to the head or the torso of the user while the user is sitting or lying on top of the article of furniture.

In some embodiments, the sensor zone may or may not be part of a bottom surface of the article of furniture. In some embodiments, the sensor zone may or may not be part of a top surface of the article of furniture. In some embodiments, the sensor zone may or may not be part of a side surface of the article of furniture.

In some embodiments, the sensor zone can comprise a sensor (e.g., a tap sensor) described herein. In some embodiments, the sensor zone can comprise one or more sensors (e.g., 1, 2, 3, 4, 5, 6, 7, 8 9, 10, or more sensors or tap sensors). The tap sensor may allow a user to control a temperature of the bed device. The tap sensor may allow a user to control a temperature of the bed device, an elevation of the bed device, an alarm of the bed device, or any combination thereof. The user may utilize a sensor zone with a tap sensor while using the bed device (e.g., while lying on the bed device).

In some embodiments, the sensor zone lies flush against a mattress or mattress cover. In some embodiments, the sensor zone does not comprise one or more buttons. In some embodiments, the sensor zone comprises one or more buttons. In some embodiments, the sensor zone comprises a touchscreen. In some embodiments, the sensor zone does not comprise a touchscreen. In some embodiments, a sensor zone described herein may be hidden behind a layer (e.g., a mattress cover). For example, the sensor zone may not be visible and/or not distinguishable from the mattress or mattress cover.

In some embodiments, the sensor zone may decrease the amount of time a user utilizes a mobile device. Decreasing the time a user utilizes a mobile device (e.g., an amount of screen time) can allow for a more restful sleep. Thus, an advantage of the bed device comprising a sensor zone described herein can be a longer night's sleep and/or a more restful sleep. A more restful sleep can be characterized by a decrease in wakefulness during a sleep session of the user. For example, the user may tap the sensor of the sensor zone to change a temperature of the bed device and thus may not have to use a mobile device to change a temperature of the bed device. The sensor zone may decrease a user's duration of time using a mobile device by at least about 1 minute, at least about 2 minutes, at least about 3 minutes, at least about 4 minutes, at least about 5 minutes, at least about 10 minutes, at least about 15 minutes, at least about 20 minutes, at least about 25 minutes, at least about 30 minutes, at least about 45 minutes, at least about 60 minutes, or greater than about 60 minutes. In some embodiments, the sensor zone may decrease a user's duration of time using a mobile device by at most about 60 minutes, at most about 45 minutes, at most about 30 minutes, at most about 25 minutes, at most about 20 minutes, at most about 15 minutes, at most about 10 minutes, at most about 5 minutes, at most about 4 minutes, at most about 3 minutes, at most about 2 minutes, at most about 1 minute, or less than about 1 minute. In some embodiments, the sensor zone may decrease a user's duration of time using a mobile device from about 1 minute to about 60 minutes. In some embodiments, the sensor zone may decrease a user's duration of time using a mobile device from about 1 minute to about 2 minutes, about 1 minute to about 3 minutes, about 1 minute to about 4 minutes, about 1 minute to about 5 minutes, about 1 minute to about 10 minutes, about 1 minute to about 15 minutes, about 1 minute to about 20 minutes, about 1 minute to about 25 minutes, about 1 minute to about 30 minutes, about 1 minute to about 45 minutes, about 1 minute to about 60 minutes, about 2 minutes to about 3 minutes, about 2 minutes to about 4 minutes, about 2 minutes to about 5 minutes, about 2 minutes to about 10 minutes, about 2 minutes to about 15 minutes, about 2 minutes to about 20 minutes, about 2 minutes to about 25 minutes, about 2 minutes to about 30 minutes, about 2 minutes to about 45 minutes, about 2 minutes to about 60 minutes, about 3 minutes to about 4 minutes, about 3 minutes to about 5 minutes, about 3 minutes to about 10 minutes, about 3 minutes to about 15 minutes, about 3 minutes to about 20 minutes, about 3 minutes to about 25 minutes, about 3 minutes to about 30 minutes, about 3 minutes to about 45 minutes, about 3 minutes to about 60 minutes, about 4 minutes to about 5 minutes, about 4 minutes to about 10 minutes, about 4 minutes to about 15 minutes, about 4 minutes to about 20 minutes, about 4 minutes to about 25 minutes, about 4 minutes to about 30 minutes, about 4 minutes to about 45 minutes, about 4 minutes to about 60 minutes, about 5 minutes to about 10 minutes, about 5 minutes to about 15 minutes, about 5 minutes to about 20 minutes, about 5 minutes to about 25 minutes, about 5 minutes to about 30 minutes, about 5 minutes to about 45 minutes, about 5 minutes to about 60 minutes, about 10 minutes to about 15 minutes, about 10 minutes to about 20 minutes, about 10 minutes to about 25 minutes, about 10 minutes to about 30 minutes, about 10 minutes to about 45 minutes, about 10 minutes to about 60 minutes, about 15 minutes to about 20 minutes, about 15 minutes to about 25 minutes, about 15 minutes to about 30 minutes, about 15 minutes to about 45 minutes, about 15 minutes to about 60 minutes, about 20 minutes to about 25 minutes, about 20 minutes to about 30 minutes, about 20 minutes to about 45 minutes, about 20 minutes to about 60 minutes, about 25 minutes to about 30 minutes, about 25 minutes to about 45 minutes, about 25 minutes to about 60 minutes, about 30 minutes to about 45 minutes, about 30 minutes to about 60 minutes, or about 45 minutes to about 60 minutes.

In some embodiments, the tap sensors are embedded in the mattress. In some embodiments, the tap sensors are embedded in the mattress cover. In some embodiments, the tap control comprises one tap, two taps, three taps, four taps, five taps, six taps, seven taps, eight taps, nine taps, ten taps, or greater than about ten taps. The sensor zone may have a memory for multiple configurations of gestures (e.g., taps). A sensor zone may control one or more functions of a bed device by a unique tap gesture (e.g., a number of taps on the sensor zone). A sensor zone may retain a memory to process at least about 1 unique tap gesture, at least about 2 unique tap gestures, at least about 3 unique tap gestures, at least about 4 unique tap gestures, at least about 5 unique tap gestures, or greater than about 5 unique tap gestures. A sensor zone may retain a memory to process at most about 5 unique tap gestures, at most about 4 unique tap gestures, at most about 3 unique tap gestures, at most about 2 unique tap gestures, or at most about 1 unique tap gesture. A sensor zone described herein can be configured to process a number of taps to execute a specific function. For example, the sensor zone comprising the tap sensor may be configured to execute the functions: (i) two taps to lower the temperature level of the bed device by one degree (e.g., 1° C.), (ii) three taps to raise the temperature by one degree (e.g., 1° C.), or (iii) any combination thereof. As another example, the sensor zone comprising the tap sensor may be configured to execute the functions: (i) two taps to lower the temperature level of the bed device by one degree (e.g., 1° C.), (ii) three taps to raise the temperature by one degree (e.g., 1° C.), (iii) four taps to adjust an elevation of the bed device (e.g., adjust an elevation from a rest position to a heightened or lowered position), or (iv) any combination thereof.

The sensor zone may control a temperature of a bed device with any number of taps (e.g., 1, 2, 3, 4, 5, or more taps). The sensor zone may control an elevation of a bed device with any number of taps (e.g., 1, 2, 3, 4, 5, or more taps). The sensor zone may control an alarm with any number of taps (e.g., 1, 2, 3, 4, 5, or more taps). For example, a user may dismiss an alarm with one or more taps. The user may snooze an alarm with one or more taps. A bed device described herein may comprise at least about 1 sensor zone (e.g., tap sensor), at least about 2 sensor zones (e.g., tap sensors), at least about 3 sensor zones (e.g., tap sensors), at least about 4 sensor zones (e.g., tap sensors), at least about 5 sensor zones (e.g., tap sensors), or greater than about 5 sensor zones (e.g., tap sensors). A bed device described herein may comprise at most about 5 sensor zones (e.g., tap sensors), at most about 4 sensor zones (e.g., tap sensors), at most about 3 sensor zones (e.g., tap sensors), at most about 2 sensor zones (e.g., tap sensors), at most about 1 sensor zones (e.g., tap sensors), or less than about 1 sensor zone (e.g., tap sensor).

The bed device may comprise two sensor zones, such that a first sensor zone is on a first side of a bed device and second sensor zone is one a second side of the bed device. In some embodiments, a user may utilize a first sensor zone and second sensor zone. In some embodiments, a first user may utilize a first sensor zone and a second user may utilize a second sensor zone. In some embodiments, the first sensor zone and the second sensor zone may have the same response(s) to tap gestures. In some embodiments, the first sensor zone and the second sensor zone may not have the same response(s) to tap gestures. For example, a first sensor zone may lower a temperature of a bed device (e.g., a first section or zone of a bed device) in response to two tap gestures and a second sensor zone may raise a temperature of a bed device (e.g., a second section or zone of a bed device) in response to two tap gestures. As another example, a first sensor zone may lower a temperature of a bed device (e.g., a first section or zone of a bed device) in response to two tap gestures and a second sensor zone may adjust an elevation of a bed device (e.g., a second section or zone of a bed device) in response to two tap gestures.

In some embodiments, a sensor zone may control one or more sections of a bed device. A user may initiate different tap gestures (e.g., unique tap gestures, e.g., number of taps) to a sensor zone to control a function of a first section of a bed device and a second section of a bed device. A first tap gesture can be different than a second tap gesture. A first tap gesture can comprise one or more taps (e.g., 1, 2, 3, 4, or more taps) and the second tap gesture can comprise one or more taps (e.g., 1, 2, 3, 4, or more taps). For example, the user may (i) contact the sensor zone with one or more taps to raise a temperature of a first section of a bed device and (ii) contact the sensor zone with one or more taps to raise a temperature of a second section of the bed device. For example, the user may (i) contact the sensor zone with one or more taps to raise a temperature of a first section of a bed device and (ii) contact the sensor zone with one or more taps to lower a temperature of a second section of the bed device. For example, the user may (i) contact the sensor zone with one or more taps to raise a temperature of a first section of a bed device and (ii) contact the sensor zone with one or more taps to raise an angular position (e.g., raise an elevation) of a second section of the bed device. For example, the user may (i) contact the sensor zone with one or more taps to raise a temperature of a first section of a bed device and (ii) contact the sensor zone with one or more taps to lower an angular position (e.g., lower an elevation) of a second section of the bed device. For example, the user may (i) contact the sensor zone with one or more taps to raise a temperature of a first section of a bed device and (ii) contact the sensor zone with one or more taps to dismiss or snooze an alarm (e.g., an alarm of the bed device or external to the bed device). For example, the user may (i) contact the sensor zone with one or more taps to lower a temperature of a first section of a bed device and (ii) contact the sensor zone with one or more taps to raise a temperature of a second section of the bed device. For example, the user may (i) contact the sensor zone with one or more taps to lower a temperature of a first section of a bed device and (ii) contact the sensor zone with one or more taps to lower a temperature of a second section of the bed device. For example, the user may (i) contact the sensor zone with one or more taps to lower a temperature of a first section of a bed device and (ii) contact the sensor zone with one or more taps to raise an angular position (e.g., raise an elevation) of a second section of the bed device. For example, the user may (i) contact the sensor zone with one or more taps to lower a temperature of a first section of a bed device and (ii) contact the sensor zone with one or more taps to lower an angular position (e.g., lower an elevation) of a second section of the bed device. For example, the user may (i) contact the sensor zone with one or more taps to lower a temperature of a first section of a bed device and (ii) contact the sensor zone with one or more taps to dismiss or snooze an alarm (e.g., an alarm of the bed device or external to the bed device). For example, the user may (i) contact the sensor zone with one or more taps to raise an angular position (e.g., raise an elevation) of a first section of a bed device and (ii) contact the sensor zone with one or more taps to raise a temperature of a second section of the bed device. For example, the user may (i) contact the sensor zone with one or more taps to raise an angular position (e.g., raise an elevation) of a first section of a bed device and (ii) contact the sensor zone with one or more taps to lower a temperature of a second section of the bed device. For example, the user may (i) contact the sensor zone with one or more taps to raise an angular position (e.g., raise an elevation) of a first section of a bed device and (ii) contact the sensor zone with one or more taps to raise an angular position (e.g., raise an elevation) of a second section of the bed device. For example, the user may (i) contact the sensor zone with one or more taps to raise an angular position (e.g., raise an elevation) of a first section of a bed device and (ii) contact the sensor zone with one or more taps to lower an angular position (e.g., lower an elevation) of a second section of the bed device. For example, the user may (i) contact the sensor zone with one or more taps to raise an angular position (e.g., raise an elevation) of a first section of a bed device and (ii) contact the sensor zone with one or more taps to dismiss or snooze an alarm (e.g., an alarm of the bed device or external to the bed device). For example, the user may (i) contact the sensor zone with one or more taps to lower an angular position (e.g., lower an elevation) of a first section of a bed device and (ii) contact the sensor zone with one or more taps to raise a temperature of a second section of the bed device. For example, the user may (i) contact the sensor zone with one or more taps to lower an angular position (e.g., lower an elevation) of a first section of a bed device and (ii) contact the sensor zone with one or more taps to lower a temperature of a second section of the bed device. For example, the user may (i) contact the sensor zone with one or more taps to lower an angular position (e.g., lower an elevation) of a first section of a bed device and (ii) contact the sensor zone with one or more taps to raise an angular position (e.g., raise an elevation) of a second section of the bed device. For example, the user may (i) contact the sensor zone with one or more taps to lower an angular position (e.g., lower an elevation) of a first section of a bed device and (ii) contact the sensor zone with one or more taps to lower an angular position (e.g., lower an elevation) of a second section of the bed device. For example, the user may (i) contact the sensor zone with one or more taps to lower an angular position (e.g., lower an elevation) of a first section of a bed device and (ii) contact the sensor zone with one or more taps to dismiss or snooze an alarm (e.g., an alarm of the bed device or external to the bed device). For example, the user may (i) contact the sensor zone with one or more taps to dismiss or snooze an alarm (e.g., an alarm of the bed device or external to the bed device) and (ii) contact the sensor zone with one or more taps to raise a temperature of a second section of the bed device. For example, the user may (i) contact the sensor zone with one or more taps to dismiss or snooze an alarm (e.g., an alarm of the bed device or external to the bed device) and (ii) contact the sensor zone with one or more taps to lower a temperature of a second section of the bed device. For example, the user may (i) contact the sensor zone with one or more taps to dismiss or snooze an alarm (e.g., an alarm of the bed device or external to the bed device) and (ii) contact the sensor zone with one or more taps to raise an angular position (e.g., raise an elevation) of a second section of the bed device. For example, the user may (i) contact the sensor zone with one or more taps to dismiss or snooze an alarm (e.g., an alarm of the bed device or external to the bed device) and (ii) contact the sensor zone with one or more taps to lower an angular position (e.g., lower an elevation) of a second section of the bed device.

A sensor zone of a bed device may comprise haptic feedback. For example, a user may tap the sensor zone and receive a vibratory feedback from the sensor zone area. This feature may provide assurance to the user that the bed device registered the tap gesture. The tap sensors of the sensor zone may operatively communicate with additional one or more sensors of the bed device. In some embodiments, the one or more sensor zones of the bed device may be positioned on a side surface of the bed device. In some embodiments, the one or more sensor zones of the bed device may be positioned on a top surface of the bed device. In some embodiments, a first sensor zone and a second sensor zone may be positioned on similar surfaces of the bed device. For example, a first sensor zone may be positioned on a side surface of a bed device and a second sensor zone may be positioned on a side of the bed device. In some embodiments, a first sensor zone and a second sensor zone may be positioned on different surfaces of the bed device. For example, a first sensor zone may be positioned on a side surface of a bed device and a second sensor zone may be positioned on a top of the bed device.

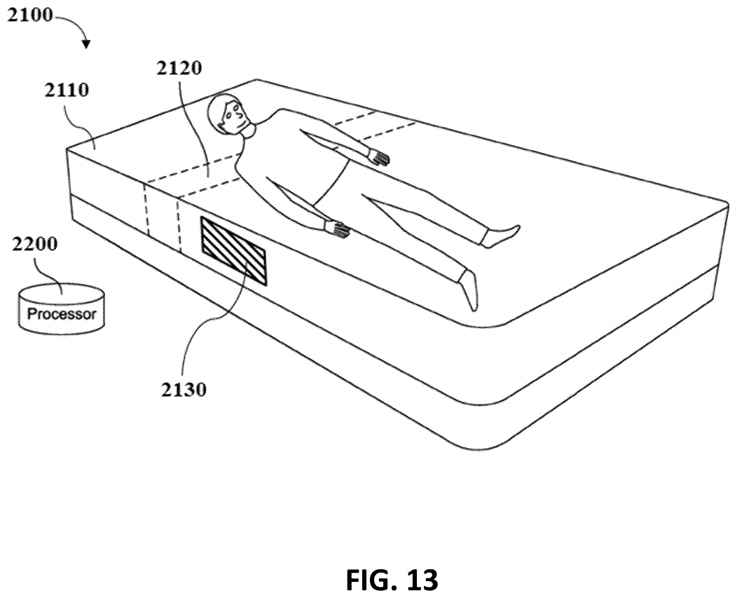

FIG. 13 schematically illustrates an example article of furniture 2100. The article of furniture 2100 can be a bed device, such as a mattress or a mattress cover. The bed device can comprise a cover layer 2110, which can be a fabric layer configured to be adjacent to or to contact the user when the user is using the article of furniture 2100. The bed device 2100 can comprise one or more sensors 2120 for detecting sensor data. The sensor data can comprise one or more biological signals associated with the user and/or one or more environmental signals (e.g., temperature, humidity, light, etc.) associated with the article of furniture 2100. For example, the one or more sensors 2120 can comprise one or more of a capacitance sensor, a pressure sensor (e.g., a piezo sensor), and/or a temperature sensor. The sensor(s) 2120 for detecting biological signal(s) can be disposed on or adjacent to a top surface of the cover layer 2110. Such sensor(s) 2120 can be disposed under the top surface of the cover layer 2110. The sensor(s) 2120 for detecting environmental signals can be disposed on or adjacent to a side surface of the cover layer 2110. Such sensor(s) 2120 can be disposed under the side surface of the cover layer 2110. The bed device 2100 can comprise a sensor zone 2130 comprising a sensor unit configured to detect and identify different interactions of the user, as provided herein (e.g., tab and/or swipe). The sensor zone 2130 can be disposed on a side surface of the cover layer 2110, such the user may not interact with the sensor unit within the sensor zone 2130 during normal use of the article of furniture (e.g., during sleeping on the top surface of the bed device). The sensor unit of the sensor zone 2130 can be disposed under the side surface of the cover layer 2110. The article of furniture 2100 can comprise (or can be operatively coupled to) a processor 2200 configured to (i) receive data/instruction from the sensor unit of the sensor zone 2130 and (ii) based on the data/instruction from the sensor unit, control (i) the condition of the article of furniture, (ii) the condition of the environment, (iii) the user device, and/or (iv) the other devices. The processor 2200 can be a part of the article of furniture 2100 (e.g., within the bed device) or disposed outside of the article of furniture 2100.

In some embodiments, the processor analyzes the data collected by the one or more sensors. In some embodiments, the bed device further comprises a temperature control unit. In some embodiments, the temperature control unit is operatively connected to the processor. In some embodiments, the processor can provide a control signal to the temperature control unit. In some embodiments, the control signal can be an instruction for the temperature control unit to change a temperature of the article of furniture. In some embodiments, the control signal can be an instruction for the temperature control unit to change a temperature of a portion of the article of furniture (e.g., bed device). In some embodiments, the control signal can be an instruction to change the temperature of the bed device based on the subject's age. In some embodiments, the control signal can be an instruction to change the temperature of the bed device based on the subject's biological sex. In some embodiments, the control signal can be an instruction to change the temperature of the bed device based on the subject's geolocation. In some embodiments, the control signal can be an instruction to change the temperature of the bed device based on the season of the subject's geolocation.

The processor can be any type of microcontroller, or any processor in a mobile terminal, fixed terminal, or portable terminal including a mobile handset, station, unit, device, multimedia computer, multimedia tablet, Internet node, cloud computer, communicator, desktop computer, laptop computer, notebook computer, netbook computer, tablet computer, personal communication system (PCS) device, personal navigation device, personal digital assistants (PDAs), audio/video player, digital camera/camcorder, positioning device, television receiver, radio broadcast receiver, electronic book device, game device, the accessories and peripherals of these devices, or any combination thereof.

In some embodiments, a surface of the sensor zone can comprise or exhibit a textured surface that is different and distinguishable (e.g., via touch or contact by the user) from other surfaces of the article of furniture (e.g., the first surface, other portions of the second surface, etc.). Such unique textured surface can permit the user to identify the sensor zone via touch or contact without having to turn the head towards the sensor zone and/or without having to look at the sensor zone. The unique textured surface can be provided by using a different material (e.g., different fabric) as compared to other surfaces of the article of furniture, different dimension (e.g., thickness, density, pattern, etc.) of a different material or the same material as compared to other surfaces of the article of furniture, etc. For example, the outer surface of the article of furniture can comprise a fabric, and the sensor zone can comprise a textured pattern (e.g., threaded pattern, polymer-printed pattern, etc.).

In some embodiments, the textured surface can comprise regulator and/or irregular topographies disposed on a surface (e.g., a fabric surface). In some embodiments, the textured surface can comprise periodical and/or ordered topographies disposed on the surface (e.g., a fabric surface), such as one or more protrusions on the surface. The topographies on the surface can comprise a dimension (e.g., a cross-sectional dimension) that is circular, triangular, square, rectangular, pentagonal, hexagonal, or any partial shape or combination of shapes thereof. The topographies can comprise a dimension (e.g., cross-sectional width, height, etc.) that is at least or at most about 0.1 millimeters (mm), at least or at most about 0.2 mm, at least or at most about 0.5 mm, at least or at most about 1 mm, at least or at most about 2 mm, at least or at most about 3 mm, at least or at most about 4 mm, at least or at most about 5 mm, at least or at most about 6 mm, at least or at most about 7 mm, at least or at most about 8 mm, at least or at most about 9 mm, at least or at most about 10 mm, at least or at most about 15 mm, or at least or at most about 20 mm.

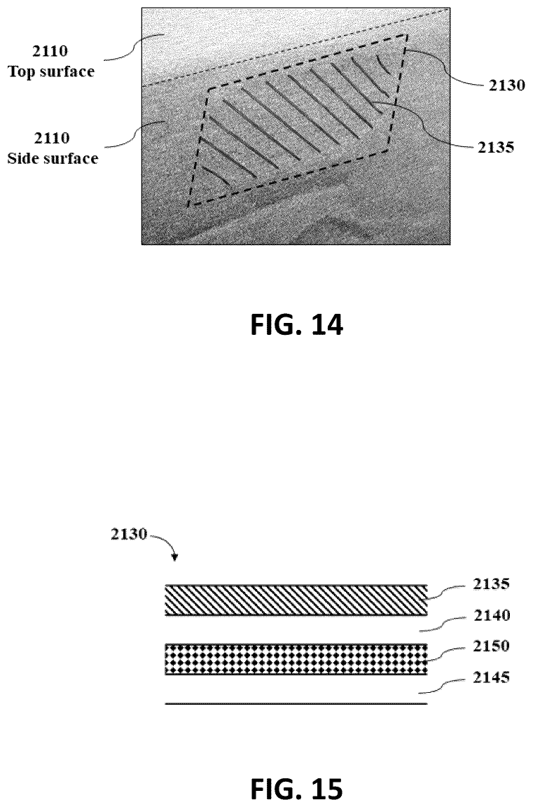

FIG. 14 illustrates a perspective view of the article of furniture 2100 shown in FIG. 13. According to FIG. 14, the side surface of the cover layer 2110 can comprise the sensor zone 2130 comprising a textured surface 2135. The textured surface 2135 can be distinguishable (e.g., by touch) from other regions of the side surface of the cover layer 2110 and other regions of the top surface of the cover layer 2110.

In some embodiments, the sensor zone can comprise a cover layer (e.g., a fabric) comprising the textured surface. The sensor unit and the textured surface can be disposed on opposite sides of the cover layer. For example, the textured surface can be disposed on an outer side the cover layer (e.g., the surface that is exposed to the environment surrounding the article of furniture), and the sensor unit can be disposed on an inner side of the cover layer (e.g., the surface that is hidden from the user or the environment surrounding the article of furniture). Accordingly, the sensor unit may be visually hidden from the user. The user may or may not be able to feel (e.g., via touch) the sensor unit disposed beneath the cover layer.

In some embodiments, the sensor zone can comprise one or more additional layers. For example, the sensor zone may comprise at least about 1 layer, at least about 2 layers, at least about 2 layers, at least about 2 layers, at least about 2 layers, at least about 2 layers, at least about 2 layers, (e.g., at least or at most about 1, at least or at most about 2, at least or at most about 3, at least or at most about 4, at least or at most about 5 additional layers, etc.) disposed adjacent to the sensor unit. The additional layer(s) can provide additional softness to the sensor zone, e.g., to reduce or prevent the user from physically feeling the hardness of the sensor unit. The additional layer(s) can be cushion pad(s). The additional layer(s) can be disposed between the cover layer and the sensor unit. Alternatively or in addition to, the additional layer(s) can be disposed on opposite sides of the sensor unit. For example, the sensor unit can be disposed between (e.g., sandwiched between) two additional layers. In another example, the additional layer(s) can comprise a pocket made of a soft material, and the sensor unit can be disposed between the pocket.

In some embodiments, the additional layer can comprise a foam. The foam can comprise a polymeric foam. Non-limiting examples of a polymeric foam can include ethylene-vinyl acetate (EVA) foam, low-density polyethylene (LDPE) foam, nitrile rubber (NBR) foam, polychloroprene foam, polyimide foam, polypropylene foam, polystyrene foam, polyurethane foam, polyurea foam, polyethylene foam, polyvinyl chloride foam, and silicone foam. For example, the foam can be a polyurethane foam. In some embodiments, the additional layer may comprise a synthetic fiberfill material. The additional layer may be a polyester fiberfill (e.g., a polyfill).

In some embodiments, the thickness of the additional layer can be at least or at most about 1 millimeter, at least or at most about 2 millimeters, at least or at most about 3 millimeters, at least or at most about 4 millimeters, at least or at most about 5 millimeters, at least or at most about 6 millimeters, at least or at most about 7 millimeters, at least or at most about 8 millimeters, at least or at most about 9 millimeters, at least or at most about 10 millimeters, at least or at most about 11 millimeters, at least or at most about 12 millimeters, at least or at most about 13 millimeters, at least or at most about 14 millimeters, at least or at most about 15 millimeters, at least or at most about 16 millimeters, at least or at most about 17 millimeters, at least or at most about 18 millimeters, at least or at most about 19 millimeters, at least or at most about 20 millimeters, at least or at most about 21 millimeters, at least or at most about 21 millimeters, at least or at most about 23 millimeters, at least or at most about 24 millimeters, at least or at most about 25 millimeters, at least or at most about 26 millimeters, at least or at most about 27 millimeters, at least or at most about 28 millimeters, at least or at most about 29 millimeters, at least or at most about 30 millimeters, at least or at most about 35 millimeters, at least or at most about 40 millimeters, at least or at most about 45 millimeters, or at least or at most about 50 millimeters.

In some embodiments, the material density of the additional layer can be at least or at most about 5 kilograms per cubic meters, at least or at most about 10 kilograms per cubic meters, at least or at most about 15 kilograms per cubic meters, at least or at most about 20 kilograms per cubic meters, at least or at most about 25 kilograms per cubic meters, at least or at most about 30 kilograms per cubic meters, at least or at most about 35 kilograms per cubic meters, at least or at most about 40 kilograms per cubic meters, at least or at most about 45 kilograms per cubic meters, at least or at most about 50 kilograms per cubic meters, at least or at most about 60 kilograms per cubic meters, at least or at most about 70 kilograms per cubic meters, at least or at most about 80 kilograms per cubic meters, at least or at most about 90 kilograms per cubic meters, or at least or at most about 100 kilograms per cubic meters. For example, the material density of the additional layer can range between about 10 kilograms per cubic meters and about 40 kilograms per cubic meters, between about 20 kilograms per cubic meters and about 30 kilograms per cubic meters, or between about 23 kilograms per cubic meters and about 28 kilograms per cubic meters.

In some embodiments, the stiffness of the additional layer, as measured by indentation load deflection (ILD) method or indentation force deflection (IFD) method, can be at least or at most about 5 ILD or IFD, at least or at most about 10 ILD or IFD, at least or at most about 15 ILD or IFD, at least or at most about 20 ILD or IFD, at least or at most about 25 ILD or IFD, at least or at most about 30 ILD or IFD, at least or at most about 35 ILD or IFD, at least or at most about 40 ILD or IFD, at least or at most about 45 ILD or IFD, or at least or at most about 50 ILD or IFD. For example, the stiffness of the additional layer can range between about 10 ILD or IFD and about 35 ILD or IFD, between about 15 ILD or IFD and about 30 ILD or IFD, or between about 20 ILD or IFD and about 25 ILD or IFD.

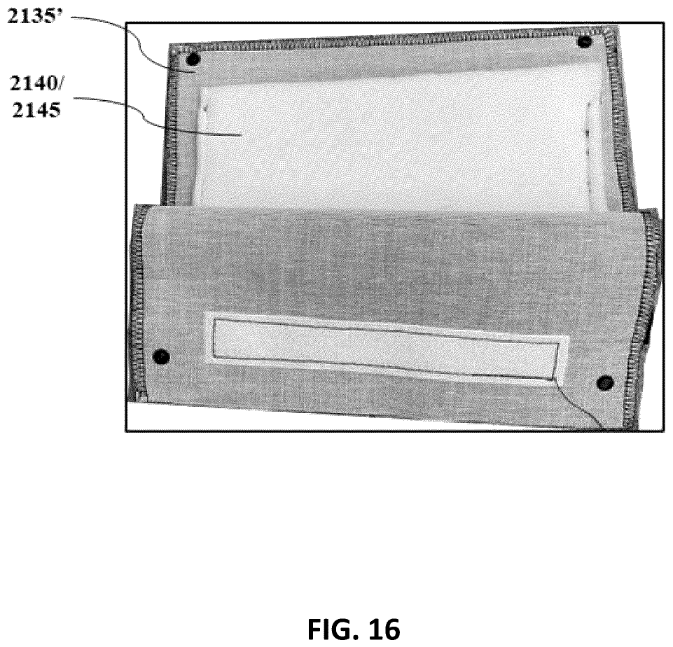

FIG. 15 schematically illustrates a cross-section of the sensor zone 2130 as shown in FIG. 13 or FIG. 14. According to FIG. 15, the sensor zone 2130 can comprise a cover layer comprising the textured surface 2135. The sensor zone 2130 can further comprise the sensor unit 2150, which sensor unit 2150 is sandwiched between two additional layers 2140 and 2145 (e.g., foam layers) to reduce the hardness of the sensor unit 2150 when the user contacts (e.g., tabs or swipes) the textured surface 2135. FIG. 16 shows an example of the sensor zone 2130 showing a side 2135′of the cover layer that is opposite the textured surface 2135. Two layers 2140/2145 made of a soft material (e.g., foam) can be attached to the side 2135′of the cover layer, such that the sensors unit 2150 can be disposed (e.g., inserted) between the two layers 2140/2145.

In some embodiments, the condition of the article of furniture can comprise temperature, vibration (e.g., to provide a massage function to the user, to enhance sleep quality of the user, to wake up the user from sleeping, etc.), shape (e.g., tile angle of at least a portion of the article of furniture, such as a bed or a chair), etc. In some cases, the article of furniture can comprise an instrument to regulate such condition of the article of furniture. The instrument can comprise at least a portion of a temperature control unit to adjust temperature of the article of furniture. The temperature control unit can comprise an electric blanket or one or more channels to permit flow of temperature-controlled fluid (e.g., liquid or air). In some cases, the instrument can comprise one or more motors to induce such vibration in at least a portion of the article of furniture. In some cases, the instrument can comprise one or more actuators to adjust the shape of the bed.

In some embodiments, the condition of the environment comprising the article of furniture can comprise ambient temperature, ambient pressure, light, noise, humidity, oxygen level, scent, etc., and such condition can be regulated by one or more instruments operatively coupled to (e.g., disposed within or adjacent to) the environment, such as a thermostat, a humidifier, an oxygen regulator, a light, a speaker, a humidifier, an electric diffuser, etc.

In some embodiments, the other devices (or instruments as used interchangeably herein) in the environment or associated with the user that can be controlled via interacting with the sensor unit of the sensor zone can include, but are not limited to, an alarm, a coffee machine, a lock, a user device (e.g., a mobile phone, a personal computer, a smart watch, etc.), a car, an exercise machine, etc.

In some embodiments, the sensor unit of the sensor zone can be configured to, upon detecting touch and/or motion of the user, (i) directly control operation of the instrument as provided herein and/or (ii) indirectly control operation of the instrument by directing a user device (e.g., a mobile device comprising a graphical user interface (GUI)) to instruct the instrument to operate accordingly. In some cases, directly controlling operation of the instrument can allow the sensor unit to bypass use of the user device (or the GUI hereof). In some cases, even when the sensor unit can directly control the operation of the instrument, the sensor unit can be configured to generate data indicate of such control in a database, and such data can be retrievable by the GUI of the user device.

Another aspect of the present disclosure provides a system comprising one or more computer processors and computer memory coupled thereto. The computer memory comprises machine executable code that, upon execution by the one or more computer processors, implements any of the methods above or elsewhere herein such as, for example, one or more operations of the sensor unit as provided herein.

In some aspects, the present disclosure provides methods for adjusting a temperature of a bed device. The methods can comprise a system described herein. In some embodiments, the methods comprise providing a bed device described herein. The bed device can comprise one or more sensors (e.g., 1, 2, 3, 4, 5, or more sensors). The bed device may comprise a temperature control unit described herein. The temperature control unit can be coupled to one or more sensors. The temperature control unit may be configured to adjust a temperature of a bed device (e.g., a portion of the bed device). The bed device can comprise a support member described herein. For example, the support member may be a foam support member. The support member may comprise a groove.

In some embodiments, the support member can comprise a plurality of grooves. For example, the support member may comprise at least about 1 groove, at least about 2 grooves, at least about 3 grooves, at least about 4 grooves, at least about 5 grooves, at least about 10 grooves, at least about 15 grooves, at least about 20 grooves, or greater than about 20 grooves. The support member may comprise at most about 20 grooves, at most about 15 grooves, at most about 10 grooves, at most about 5 grooves, at most about 4 grooves, at most about 3 grooves, at most about 2 grooves, at most about 1 groove, or less than about 1 groove. In some embodiments, the support member may comprise from about 1 groove to about 30 grooves. In some embodiments, the support member may comprise from about 1 groove to about 2 grooves, about 1 groove to about 3 grooves, about 1 groove to about 4 grooves, about 1 groove to about 5 grooves, about 1 groove to about 8 grooves, about 1 groove to about 10 grooves, about 1 groove to about 15 grooves, about 1 groove to about 20 grooves, about 1 groove to about 30 grooves, about 2 grooves to about 3 grooves, about 2 grooves to about 4 grooves, about 2 grooves to about 5 grooves, about 2 grooves to about 8 grooves, about 2 grooves to about 10 grooves, about 2 grooves to about 15 grooves, about 2 grooves to about 20 grooves, about 2 grooves to about 30 grooves, about 3 grooves to about 4 grooves, about 3 grooves to about 5 grooves, about 3 grooves to about 8 grooves, about 3 grooves to about 10 grooves, about 3 grooves to about 15 grooves, about 3 grooves to about 20 grooves, about 3 grooves to about 30 grooves, about 4 grooves to about 5 grooves, about 4 grooves to about 8 grooves, about 4 grooves to about 10 grooves, about 4 grooves to about 15 grooves, about 4 grooves to about 20 grooves, about 4 grooves to about 30 grooves, about 5 grooves to about 8 grooves, about 5 grooves to about 10 grooves, about 5 grooves to about 15 grooves, about 5 grooves to about 20 grooves, about 5 grooves to about 30 grooves, about 8 grooves to about 10 grooves, about 8 grooves to about 15 grooves, about 8 grooves to about 20 grooves, about 8 grooves to about 30 grooves, about 10 grooves to about 15 grooves, about 10 grooves to about 20 grooves, about 10 grooves to about 30 grooves, about 15 grooves to about 20 grooves, about 15 grooves to about 30 grooves, or about 20 grooves to about 30 grooves.

The grooves may house a channel. The one or more grooves of the support member may house a plurality of channels. The plurality of channels may rest within the grooves such that a channel may not be raised above the groove. A layer resting on top of the support member may be capable of resting flat with the plurality of channels housed in the grooves. The plurality of channels can be configured for fluid flow. The channels housed in the grooves may provide for a more consistent fluid flow.

In some embodiments, the method may comprise detecting at least one biological signal for a user. The user may be using the bed device described herein. In some embodiments, the detecting may occur by using the one or more sensors of the bed device. A processor may receive the detected one or more biological signal. Based on the detected signal, the method can comprise using the temperature control unit to adjust a temperature. A temperature of the fluid of the channels may be adjusted. Adjusting the temperature of the fluid may adjust a temperature of the bed device (e.g., portion of the bed device).

As an example, provides herein is a method for adjusting a temperature of a bed device, comprising: (a) providing the bed device comprising: (i) one or more sensors; (ii) a temperature control unit coupled to the one or more sensors, wherein the temperature control unit is configured to adjust a temperature of a portion of the bed device, and (iii) a support member comprising a plurality of grooves, wherein the grooves are configured to house a plurality of channels configured for fluid flow; and (b) detecting, via the one or more sensors, at least one biological signal from a user of the bed device; (c) configuring a processor to receive the detected at least one biological signal; and (d) based on the detected at least one biological signal, using the temperature control unit to adjust temperature of the fluid of the plurality of channels to thereby adjust the temperature of the bed device.

The support member can comprise a plurality of junctions. A junction of the plurality of junctions may be a junction as described herein. The plurality of grooves of the support member may be separated into sections. For example, a support member may comprise a first section with a first set of grooves, a first plurality of channels, or any combination thereof. A support member may comprise a second section with a second set of grooves, a second plurality of channels, or any combination thereof. The first plurality of channels and second plurality of channels may comprise different fluid flows. In some embodiments, the first plurality of channels and second plurality of channels may comprise same fluid flows. The fluid can comprise a liquid. The fluid can comprise a gas. In some embodiments, the temperature control unit is disposed adjacent to the plurality of channels. In some embodiments, the temperature control unit is disposed underneath the plurality of channels.

As another example, provided herein is a method comprising: (a) providing the bed device comprising: (i) one or more sensors; (ii) a temperature control unit coupled to the one or more sensors, wherein the temperature control unit is configured to adjust a temperature of a por-tion of the bed device, and (iii) a sensor zone positioned on a bed device, wherein the sensor zone is con-figured to control one or more functions of the bed device based on a contact from the user; and (b) detecting, via the one or more sensors, at least one biological signal from a user of the bed device; (c) configuring a processor to receive the detected at least one biological signal; and (d) based on the detected at least one biological signal, using the temperature control unit to adjust temperature of the fluid of the plurality of channels to thereby adjust the temperature of the bed device. The sensor zone can comprise a sensor zone as described herein.

### Computer Systems

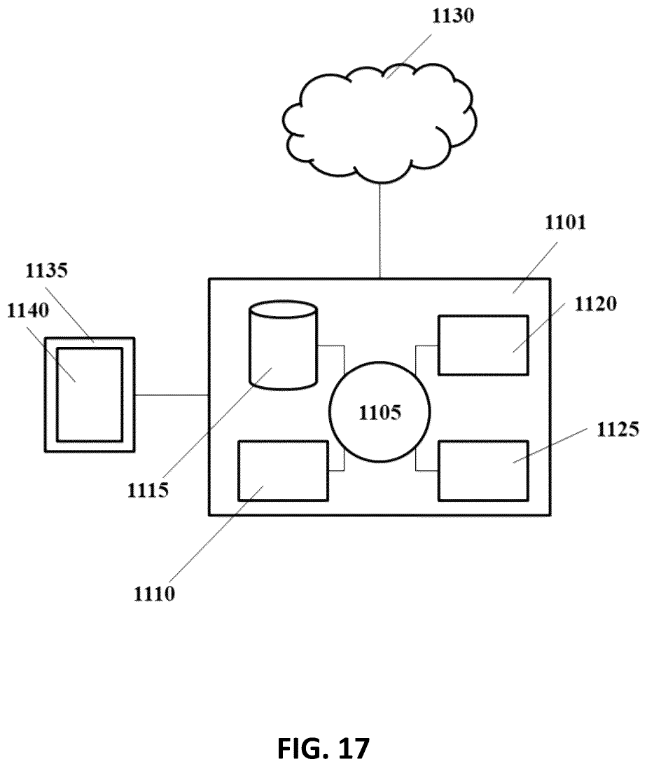

The present disclosure provides computer systems that are programmed to implement methods of the disclosure. FIG. 17 shows a computer system 1101 that is programmed or otherwise configured to direct operation of the sensor unit or other devices/instruments operatively coupled thereto. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, distinguish whether a sensing signal detected by the sensor unit of the sensor zone is associated with a direct contact (e.g., tab, swipe, etc.) from the user of the article of furniture.

Systems and methods of the present disclosure may be combined with or modified by additional systems comprising an article of furniture (e.g., a bed device) and methods of use thereof. For example, systems and methods of detecting a biological signal or a condition (e.g., a sleep disorder) of a user of an article of furniture, regulating a temperature or configuration of an article of furniture, regulating a biological signal or condition (e.g., a sleep disorder) of the user on an article of furniture, regulating operation of other devices operatively coupled to the article of furniture are described in U.S. Patent Publication No. 2015/0351556 (“BED DEVICE SYSTEM AND METHODS”), U.S. Patent Publication No. 2016/0128488 (“APPARATUS AND METHODS FOR HEATING OR COOLING A BED BASED ON HUMAN BIOLOGICAL SIGNALS”), U.S. Patent Publication No. 2017/0135882 (“ADJUSTABLE BEDFRAME AND OPERATING METHODS FOR HEALTH MONITORING”), U.S. Patent Publication No. 2017/0135632 (“DETECTING SLEEPING DISORDERS”), U.S. Patent Publication No. 2020/0405998 (“SLEEP POD”), and U.S. Patent Publication No. 2021/0315389 (“SYSTEMS AND METHODS FOR REGULATING A TEMPERATURE OF AN ARTICLE OF FURNITURE”), each of which is incorporated herein by reference in its entirety.

### EXAMPLES

### Example 1. Fluid Channels Laid into Grooves of a Support Member

Different tubes of different materials were made and assessed for their compatibility for use as part of an article of furniture (e.g., a bed) as illustrated in FIG. 1. A tube made of thermoplastic polyurethane (TPU) was durable and sufficient for puncture resistance, tear resistance, and chemical resistance. Such characteristic(s) of the TPU-based tube was better than the characteristic(s) of a tube made of other materials, such as rubber or silicone.

In some cases, some TPU-based tubes were stiffer and less comfortable for a user than a tube made of polystyrene thermoplastic elastomer (TPS), such as poly(styrene-b-ethylene/butylene-b-styrene) (SEBS) exhibiting hardness of less than 70 (e.g., less than or equal to 60, less than or equal to 50, less than or equal to 45, etc.) in accordance with the Shore A hardness scale.

Thus, in some cases, tubes made of TPS may exhibit sufficient durability, puncture resistance, tear resistance, chemical resistance, and softness to be utilized for the article of furniture as provided herein. However, in some cases, tubes made of TPU may be sufficient. Alternatively or in addition to, a tube made of polyvinyl chloride (PVC) may be stiffer than the tube made of the TPS, but may be a viable candidate for the article of furniture.

### Example 2: A Hybrid Channel

As provided herein, a channel (e.g., a tube) for providing fluid flow can be a hybrid channel comprising a plurality of channel layers, e.g., an outer channel layer surrounding an inner channel layer.

For example, the hybrid channel can comprise (i) an outer channel layer made of TPU and (ii) an inner channel layer made of TPS (e.g., SEBS), to combine the chemical resistance and puncture resistance of TPU on the outside while preserving the softness of TPS. The thickness of the outer channel layer (e.g., 0.1 millimeters) can be thinner than the thickness of the inner channel layer (e.g., 0.75 millimeters).

In another example, the hybrid channel can comprise (i) an outer channel layer made of TPU and (ii) an inner channel layer made of PVC, to combine the chemical resistance and puncture resistance of TPU on the outside while preserving the softness of PVC (e.g., relative to TPU). The thickness of the outer channel layer (e.g., 0.1 millimeters) can be thinner than the thickness of the inner channel layer (e.g., 0.5 millimeters).

### Example 3. Tap to Control Operations of an Article of Furniture

In some embodiments, the article of furniture as provided herein can comprise a sensor zone on a surface of the article of furniture (e.g., the sensor zone 2130 as schematically illustrated in FIG. 13), wherein a user of the article of furniture (e.g., while on the article of furniture) can interact with the sensor zone (e.g., tab or swipe the sensor zone) to control one or more conditions of the article of furniture (e.g., a temperature of a bed device, a configuration such as incline or decline of a bed device, etc.) and/or an environment the article of furniture.

### Example 4: Tap Zones of Bed Device

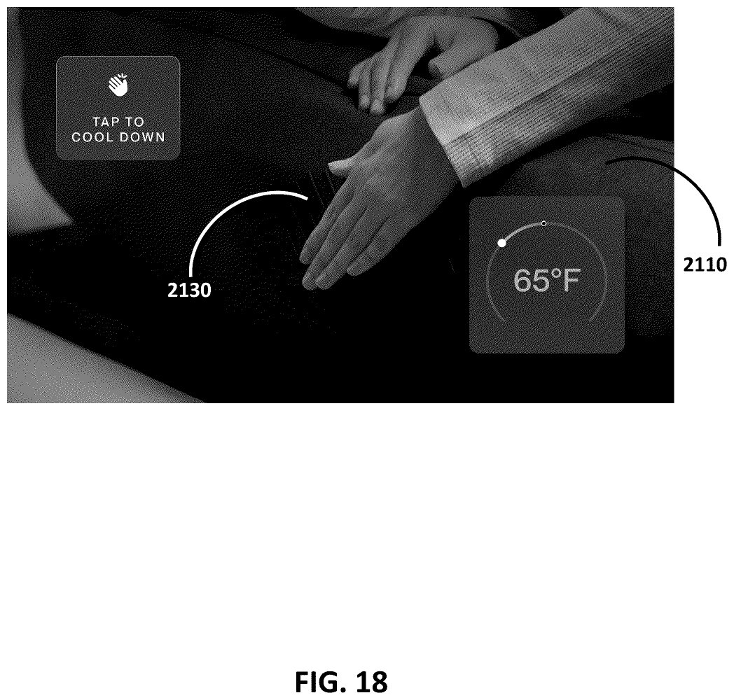

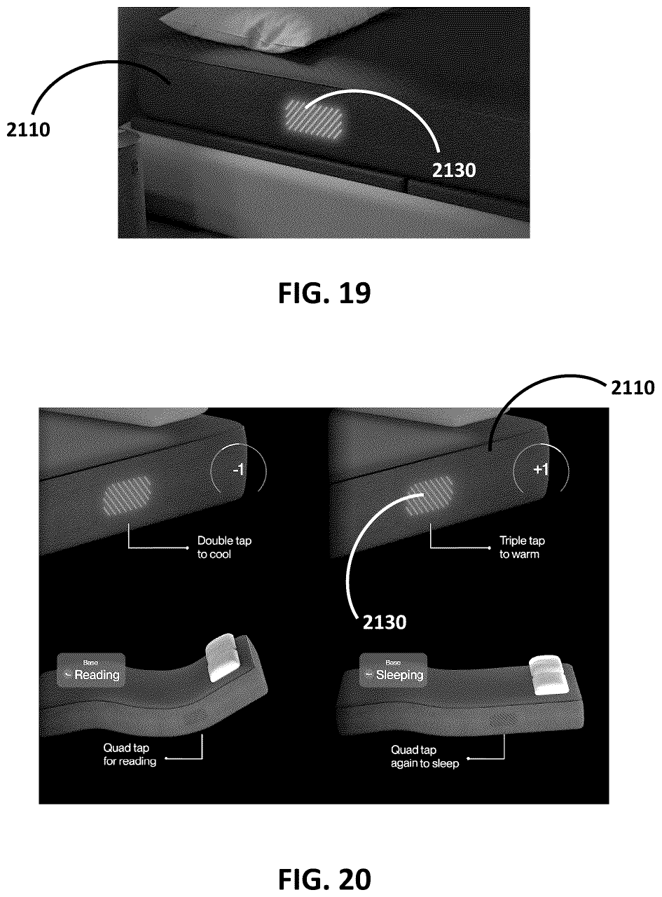

A sensor zone (e.g., tap control) feature was developed for controlling a bed device without relying on a phone. This can, in turn, reduce nighttime screen exposure. To solve this problem, an innovative sensor solution was designed. The sensor was a tap sensor called: Tap to Control (FIG. 18). The tap sensor featured two distinct tap zones, one on each side of the bed device (FIG. 19). The tap sensors allowed a user to control the temperature, base elevation, and alarm without ever moving from the bed.

### Development of the Tap Sensors

As screen time at night can negatively impact the quality of sleep, a new way of controlling bed device was developed so a user may not have to use an app on a mobile device. Considering that the controls should be simple and intuitive, a version of the bed device was built with tap sensors embedded directly into the mattress cover. With a simple double or triple tap, a user can control the Pod's thermal or elevation controls, as well as the alarm.

### How to Use Tap to Control

Just like the controls on a pair of wireless headphones, the bed device (Pod 4) featured two tap zones that responded according to a pre-set series of gestures. Two taps lowered the temperature level of the Pod by one, while three taps raised the temperature by one. Four taps can be used on Pod 4 Ultra to switch between the Base's Reading and Sleeping positions.

Users also have control over their alarms via Tap to Control, allowing them to snooze or dismiss it with just two taps (a setting that can be pre-determined within the app).

### Overview of the Overnight Gestures

There are multiple tap gestures that can used to initiate different functions of the bed device (FIG. 20). For example, two taps to decrease Pod temperature by one level, three taps to increase Pod temperature by one level, and four taps to switch between reading elevation and sleeping elevation of the base. Alarm gestures included: two taps to snooze or dismiss the user's alarm.

### The Technology Behind Tap to Control

Tap to Control utilizes vibration and proximity sensors within the mattress Cover. There are two tap zones powered by these sensors, one on each side of the Cover, so that two users can share a Pod and can access a tap zone from their side. As a user taps on these zones, a real-time algorithm reads the tap patterns live and responds based on each command. To differentiate intentional taps from normal movements and accidental bumping, the sensors inside the tap zones work in conjunction with the sensors across the mattress Cover to ensure only purposeful adjustments are made.

This phone-free solution is fully integrated into the Pod so it's always exactly where a user left it, whereas a remote can be misplaced or left across the room. The Pod's tap zones can be just an arm's reach away when a user is settled in bed, ultimately enhancing a relaxation experience.

To ensure that the tap zones were consistently responsive to users, a variety of tests were conducted to see exactly where people were most likely to tap on the sides of their bed, as well as how they were tapping. Data of night sessions was collected to develop a streamlined algorithm for tap detection, which can be adjusted according to how hard you tap. To ensure clear communication between a user and bed device, haptic feedback echoes user's taps back to the user to confirm the pattern before adjusting.

The tap sensor feature, Tap to Control, can effectively lowered overnight phone usage by at least 10% as users won't have to reach for their device to adjust the temperature or elevation.

While preferred embodiments of the present disclosure have been shown and described herein, it will be obvious to those skilled in the art that such embodiments are provided by way of example only. It is not intended that the present disclosure be limited by the specific examples provided within the specification. While the present disclosure has been described with reference to the aforementioned specification, the descriptions and illustrations of the embodiments herein are not meant to be construed in a limiting sense. Numerous variations, changes, and substitutions will now occur to those skilled in the art without departing from the scope of the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein can be employed in practicing the present disclosure. It is therefore contemplated that the present disclosure shall also cover any such alternatives, modifications, variations or equivalents. It is intended that the following claims define the scope of invention and that methods and structures within the scope of these claims and their equivalents be covered thereby.

What is claimed is:

1. A system comprising:
(a) a bed device comprising:
(i) one or more sensors for detecting at least one biological signal from a user of the bed device,
(ii) a temperature control unit coupled to the one or more sensors, wherein the temperature control unit is configured to adjust a temperature of a portion of the bed device, and
(iii) a sensor zone positioned on a bed device, wherein the sensor zone is configured to control one or more functions of the bed device based on a contact from the user, wherein the sensor zone comprises a first sensor zone and a second sensor zone; and
(b) a processor operatively coupled to the temperature control unit and the one or more sensors.

2. The system of claim 1, wherein the first sensor zone, the second sensor zone, or both are positioned on a side surface of the bed device or a top surface of the bed device.

3. The system of claim 1, wherein the one or more functions comprise a control for a temperature of the bed device.

4. The system of claim 1, wherein the one or more functions comprise a control for an elevation of the bed device.

5. The system of claim 1, wherein the one or more functions comprise a control for an alarm of the bed device.

6. The system of claim 1, wherein each of the one or more functions of the bed device are controlled by a unique tap gesture on the sensor zone.

7. The system of claim 6, wherein the sensor zone is configured to process a plurality of unique tap gestures.

8. The system of claim 6, wherein the sensor zone is configured to process at least four unique tap gestures.

9. The system of claim 1, wherein the first sensor zone controls one or more functions of a first section of the bed device, and wherein the second sensor zone controls one or more functions of a second section of the bed device.

10. The system of claim 9, wherein the first sensor zone controls the one or more functions of the first section of the bed device based at least in part on a contact from a first user on the first section of the bed device, and wherein the second sensor zone controls the one or more functions of the second section of the bed device based at least in part on a contact from a second user on the second section of the bed device.

11. The system of claim 9, wherein the one or more functions of the first section of the bed device comprises a temperature of the first section of the bed device, and wherein the one or more functions of the second section of the bed device comprises an elevation of the second section of the bed device.

12. The system of claim 1, wherein the first sensor zone, the second sensor zone, or both is configured to provide haptic feedback to the user.

13. The system of claim 1, wherein the bed device is a mattress.

14. The system of claim 13, wherein the first sensor zone, the second sensor zone, or both are embedded within the mattress.

15. The system of claim 1, wherein the bed device is a mattress cover.

16. The system of claim 15, wherein the first sensor zone, the second sensor zone, or both are embedded within the mattress cover.

17. The system of claim 1, wherein the first sensor zone, the second sensor zone, or both comprise a button.

18. The system of claim 1, wherein the first sensor zone, the second sensor zone, or both do not comprise a touchscreen.

19. The system of claim 1, wherein the first sensor zone, the second sensor zone, or both comprise a unique surface texture as compared to a different surface of the bed device.

20. The system of claim 1, wherein the first sensor zone, the second sensor zone, or both comprise an accelerometer.

21. The system of claim 1, wherein the processor is configured to control the one or more functions of the bed device based on both (i) the contact from the user on the sensor zone and (ii) the detected at least one biological signal from the user.
